SEQUENCE LISTING (19 sequences)

SEQ ID NO: 1 - DNA, 6143 bp, Hypocrea jecorina
atggacccggacccggacctgcacctgccagcacgagtagagcgcgcgagcctggatggtagcgtgtttgtagcccacgatgcgcatccgccggctcgccatctgccccatctccatctcccccagctggcccatccagcctcaaactcaacctccaacttcaccttgcgctcgcactcgcactcgcccgctgccggcgacgtcgtcctgcccgcctcaaccgccaatgcccacgtcctcgcccaccatgcagctgccaccctagatcctgacgactactacaggagctacgagacagcctccagccccggcggcgagccacaatcgctcccaatggcctcgccagtcccgcctcccacgccggatggggctgctgcaagacacctgcagccgcctgcaaatcgcagtgctggccgtcagggtaccagtaataccgccgtgcgctccgcctcgagccccctcgaccgcaggccagccgcggcggcgccgtcgtcaagatccccctcggccagcgcccccagcgtgagggatctgaagaagcgcttcgatcagaacagcggccccaactccatcccaagagctcctccacgccaggctgcaccggtggcccgggtcagcaagcaagagtccagcaaacccaagcccaagcccaagcccagcctgtcggccccttcgtccacgcacccaggctctcgctcggcggtccccgcttccaactcgtccaagcaatctcggtcgtcgtcgtcgtccaactcagcctcgacctcgcgctcgacgtcgacgccggccccgtcagcccgctcgcagaagccacggcacgttgagcgcgagcaggcggcgggaagttcgcggtcgtttgcaaaccgtattgggaaccatcagcctgagacatccactactaacgggaactcgaacgcctccagctctatgacaccacgcccacacaagtccccaccctcagcctcccagccgtcgccgccatccccaaagtctaatcctcagttcccgggcctcctgtttggcgaaatccccccgggccagctcgacgttgccgcgccaggctttggcatcgacaacatccgcccgcgacgaacgtccgaatccaacgtgcacggcctcgcggggcgtcaacgcagcctctccgacctcgaagccgagcccgcgtccccgtccagctggtacagggacgtccacatgtcccgagggagcacgccgaaaacccacgcgcgatctcgcagcgatctctcggcgctgaagcccatcctcacaaccttgcatcccaagtcgcctacgcccgcatcggcgtcggcatcagcaaagacaagggcgaaatcaccaacagtagctgctttcgcggcagccacggtaacggcaacggctgtcgcagcaggggccgcggccgccaccccttcggtctcgcacgcgtcggcagccgccagctccaaactccccgtttccatcagcaggaggcttgcgagcccaaccaactccagtcctacctcttcgcgatcagcctcgccctctgcgcttcgcagactgccagccaatggtcgtagcaatagcagacaggccaaaccgatcgcccccacgaaccgagcgaaaacacccacgcagacgggtcgaaagcagcctccccaggggctcgttacaccgagtaatagcaacaaccggctccaagcgtacgtgtccaccgcaccacccaagctctcaccgccattgaggagctctcgaccgcgacagccagtctccgtagcgacgacagcaagctcccggatgaaagaggcggcggccaaggcgaagccccctgcacggccaaatgcacgggacggcccttcgtcgtccaagacgtacgatccggcgaagcgtaaaatcatagtgggacccatagacttcgagcagcgtcgggaacacattcgactggcatacaccaagaccattcgcgaaagccaggcctttgaggtgcgccagaaagcggtggagaggaggcggaagcagatggaggaggccaaatcggaagccgcagccaccgaagctacagcgacaagtagcacagctacaggcccatctacgtctgcggattcaccgaccacggtgactgctggagatgacgaggcgacgaataaggggccctcggacggccatgcacgagtaattccagaagtaatagcggcagagacagggctggtcgcgagcccctcggcacaggagccggctgtggaactcccagctgatcatggctctcgcgctgctgtggagaaccagggaacccttctagaaatcccgacagatgcagcctcgcacccggcatctggcaagggtgatgattcgccgacgctgggcatccccgggggcttcccagaaccctcaccgtcctccgccgcggccagaacgcagcggcctctgtcgaccatttcaatcatttcagccacgtctgctgttacggagtttgatacagagccccaggcggagctcgccgacctggctatgtccgatcggccgctcagcccccagatcatagtgccgacgagggagcgctctcagtatagaagcccctttgaggacgacgatttcccatcgtcgccgcctcggccgcgtcctgcggcccatccccatcaaatcagccaagatcgccaccatatcgaccagccgttcatccctgacgcttattacgacaatgagcatcgggagcaccccttggagacccatgcccagcaagattaccaaaccattgtgacgattctgccgcagccctcacgcgagccccctgctgccgaggagactgcgcaggcggcatcttttccccgactagacattcaagatgaatctgactgccactcggatctagaaagcgttccggcaatggctcgtcgcatgcgtagtgatgtcgacgatgccgccactgatgcctgcaccgaagagacggacgaccgcgatgggatggaggacgagcggtccccgtaccgctatggcggcaccctctcgtcgaacagggcctctacatgcgcatcatcggacatagaaacgtttgacgaccttttgtacccttcgcatgacgagcagctggatgcgggcccaccaaacagactactggcgccgccctcgatctctcgggccgacagatcgagccaccagaccgcatggacgaatgtctccgtcgaaagcattacgcgctccgaggcttcagactctcctgttctacgagccagctcctggaagatgtccggttcgtccgggcgagacgactcttccctcagaagcctcccctatagcggagtcggcgtccgcccttcagtcgactcgacgcggtcgtcgattcagcttggccagcagctcccggagctagacacgggcgaaggcttctccatcccgtatctgtccgcagaggcaacctccgacctctcctacctctcttcccccgggaaacacgagccagcacctcacccgcgctctggacggaactcggccatcgactcgcaaacgtcgagcgtgttctatgagcagtcccagtatggcagcacgttggtcaactccgatcgcgggagtggagaatatgtctcccaccattcggaaactcctctgtcgatgatggacttgacctcgatggagactatggatcggtactatgacggtcgcacccaggtggatagcgatgccaagtcattcatccaagaatccgaggggctaagcagcgaagagcggcatcgccttatccagaggcgtaacgtcatcaaggagcttgtggataccgaagcaatttttgtaagagacatgaacattgtggaggaaatctacaagggcaccgcagaggcgtgccccaagctggacaccaagattgtcaagctcatcttcaggaacagcgacgagattatcgagtttcacacctcgttcctcgtcctcctcaaagaggcagtggccagcatttacgtaccgaagggcggccggtctctcgtcgcgagagaagactccatctattcggaacaaggccagacttccatcgtcgacctcagcgatgccaaggacagagagacgtcgctcggtccaactttccaagccaacatggagaagatgaagcttgctcacgaagggttcctgcgaaacagcgaccaagcagcaaagaaactgatccagatccagcaggacccgacggtgcagatatggctgaacgagtgtaatgaggtagccaaggacctgacagctgcctgggatctggactcccttctcatcaagccgatgcaacgaatcacaaagtatccgaatctgatcatgacgctccttcagcacacgccccaggaccaccccgatcgggaggccctcgtgatggccaaagaggcgctcgaagaggccattatcgagatcaacaagacgaagaagaactttgagctggtcggacagatcgtcggtagaaaacgtaaggagtccgacgtgaaggccggactcgctcgtgcctttggcaagaaggtggacaagctgcaaggcggaactcggccaccggaggatccagaatatctcaagctggaggaaaggttcagcgacgattacttgcggctacaggtcgtcttgcgcgatgtcgagttctacacccggcaagtctcatcgtatgtgcacgagttcctgcagtacctatcagccatcgagctggtcatgcgtcttcagccgggcagcttccccgagctggagagcaagtgggtgcgcttcaatatctccattcgcgacattgagaaggtagcgctcgagcagcatgtaagcttcgcaaacctccctttcccttgccccccctaaagcaaattttcctcgcaaaggactatactgactttctattgcaacagctgtcacagattcgaaagcatgtcattgagccttttgagcaggtcatcaagtcctacgggaacccctcgctggctatgaagaagagacaaaagcgccgagtcgtgtgggagcgcgcagagcagctgaagaaggcaggcaaaagcgtcgatcccaagcttaaggagctggttgagcagtatgaagctctcaacgatacgctaatcaaggagcttcccaagctttcggcgctgacggagaaggtgggaaacatttgtctcagcaatctcatcaacatccaggccaattggtacttcatttggagggagaagatgagagctgtgctgccggactcgcccacgatgccagacattgaggaaatagtctcgactttccagcgagactttccctatgcgaacgagatgatggccagcatcggcatcatcaacccagcctaccgcggaaggacatcacaatcaacgaaccacggagacgacgccggcctgcccaggactagaggccgaacctcagagtcggtagataggggatggagtcagtctttcaacggcgaaggcgcaccaagcctaccgcctcccgattttggcaaacggcatagcggctcgtttaccctctcccccatcagcgccggcccgtcctcctctggcttcgggacgtcagctcccagtccccaccagtactattatcgtgacttttatgccggcctgtcaagcaaccaggcgggaatgacatccccgagatcggccgaggtaccagcaacctctcgatcgctcggaggcacgcggccgagtacgggcaaaagctacgattcgtcggcaatctcaatgccgagacagagcacggagtcggcgcctcacatccgacgggactcgggcacggcgtactattccagctaccaccagcacgacagccgtagattctcaaatctctttcactctgcccttcctctaccggacggccccgaagagagtcagcggtcctctagagcatcatcgcgggagcgagcacatgcttctgatgggtacaatatcctatggctggcagcgtcgctcttcgagtttaacatctcgacaaccaagcacgaggctggctatccttatctgacgtatcaggccggcgaggtatgtttttttttttttctagtatcccagagctcgcaccgtctcttgtggagctagacatgctgacaatgagaatcttgacagatattcgatgtaattgccgagaaaggcgagctttggcttgccaagaatcaagacgacccaacggaccaggtgggctggatctggtccaaacactttgcgaaattggccgactcatag

SEQ ID NO: 2 - protein, 1990 aa, Hypocrea jecorina
MDPDPDLHLPARVERASLDGSVFVAHDAHPPARHLPHLHLPQLAHPASNSTSNFTLRSHSHSPAAGDVVLPASTANAHVLAHHAAATLDPDDYYRSYETASSPGGEPQSLPMASPVPPPTPDGAAARHLQPPANRSAGRQGTSNTAVRSASSPLDRRPAAAAPSSRSPSASAPSVRDLKKRFDQNSGPNSIPRAPPRQAAPVARVSKQESSKPKPKPKPSLSAPSSTHPGSRSAVPASNSSKQSRSSSSSNSASTSRSTSTPAPSARSQKPRHVEREQAAGSSRSFANRIGNHQPETSTTNGNSNASSSMTPRPHKSPPSASQPSPPSPKSNPQFPGLLFGEIPPGQLDVAAPGFGIDNIRPRRTSESNVHGLAGRQRSLSDLEAEPASPSSWYRDVHMSRGSTPKTHARSRSDLSALKPILTTLHPKSPTPASASASAKTRAKSPTVAAFAAATVTATAVAAGAAAATPSVSHASAAASSKLPVSISRRLASPTNSSPTSSRSASPSALRRLPANGRSNSRQAKPIAPTNRAKTPTQTGRKQPPQGLVTPSNSNNRLQAYVSTAPPKLSPPLRSSRPRQPVSVATTASSRMKEAAAKAKPPARPNARDGPSSSKTYDPAKRKIIVGPIDFEQRREHIRLAYTKTIRESQAFEVRQKAVERRRKQMEEAKSEAAATEATATSSTATGPSTSADSPTTVTAGDDEATNKGPSDGHARVIPEVIAAETGLVASPSAQEPAVELPADHGSRAAVENQGTLLEIPTDAASHPASGKGDDSPTLGIPGGFPEPSPSSAAARTQRPLSTISIISATSAVTEFDTEPQAELADLAMSDRPLSPQIIVPTRERSQYRSPFEDDDFPSSPPRPRPAAHPHQISQDRHHIDQPFIPDAYYDNEHREHPLETHAQQDYQTIVTILPQPSREPPAAEETAQAASFPRLDIQDESDCHSDLESVPAMARRMRSDVDDAATDACTEETDDRDGMEDERSPYRYGGTLSSNRASTCASSDIETFDDLLYPSHDEQLDAGPPNRLLAPPSISRADRSSHQTAWTNVSVESITRSEASDSPVLRASSWKMSGSSGRDDSSLRSLPYSGVGVRPSVDSTRSSIQLGQQLPELDTGEGFSIPYLSAEATSDLSYLSSPGKHEPAPHPRSGRNSAIDSQTSSVFYEQSQYGSTLVNSDRGSGEYVSHHSETPLSMMDLTSMETMDRYYDGRTQVDSDAKSFIQESEGLSSEERHRLIQRRNVIKELVDTEAIFVRDMNIVEEIYKGTAEACPKLDTKIVKLIFRNSDEIIEFHTSFLVLLKEAVASIYVPKGGRSLVAREDSIYSEQGQTSIVDLSDAKDRETSLGPTFQANMEKMKLAHEGFLRNSDQAAKKLIQIQQDPTVQIWLNECNEVAKDLTAAWDLDSLLIKPMQRITKYPNLIMTLLQHTPQDHPDREALVMAKEALEEAIIEINKTKKNFELVGQIVGRKRKESDVKAGLARAFGKKVDKLQGGTRPPEDPEYLKLEERFSDDYLRLQVVLRDVEFYTRQVSSYVHEFLQYLSAIELVMRLQPGSFPELESKWVRFNISIRDIEKVALEQHLSQIRKHVIEPFEQVIKSYGNPSLAMKKRQKRRVVWERAEQLKKAGKSVDPKLKELVEQYEALNDTLIKELPKLSALTEKVGNICLSNLINIQANWYFIWREKMRAVLPDSPTMPDIEEIVSTFQRDFPYANEMMASIGIINPAYRGRTSQSTNHGDDAGLPRTRGRTSESVDRGWSQSFNGEGAPSLPPPDFGKRHSGSFTLSPISAGPSSSGFGTSAPSPHQYYYRDFYAGLSSNQAGMTSPRSAEVPATSRSLGGTRPSTGKSYDSSAISMPRQSTESAPHIRRDSGTAYYSSYHQHDSRRFSNLFHSALPLPDGPEESQRSSRASSRERAHASDGYNILWLAASLFEFNISTTKHEAGYPYLTYQAGEIFDVIAEKGELWLAKNQDDPTDQVGWIWSKHFAKLADS

SEQ ID NO: 3 - DNA, 5973 bp, Hypocrea jecorina
atggacccggacccggacctgcacctgccagcacgagtagagcgcgcgagcctggatggtagcgtgtttgtagcccacgatgcgcatccgccggctcgccatctgccccatctccatctcccccagctggcccatccagcctcaaactcaacctccaacttcaccttgcgctcgcactcgcactcgcccgctgccggcgacgtcgtcctgcccgcctcaaccgccaatgcccacgtcctcgcccaccatgcagctgccaccctagatcctgacgactactacaggagctacgagacagcctccagccccggcggcgagccacaatcgctcccaatggcctcgccagtcccgcctcccacgccggatggggctgctgcaagacacctgcagccgcctgcaaatcgcagtgctggccgtcagggtaccagtaataccgccgtgcgctccgcctcgagccccctcgaccgcaggccagccgcggcggcgccgtcgtcaagatccccctcggccagcgcccccagcgtgagggatctgaagaagcgcttcgatcagaacagcggccccaactccatcccaagagctcctccacgccaggctgcaccggtggcccgggtcagcaagcaagagtccagcaaacccaagcccaagcccaagcccagcctgtcggccccttcgtccacgcacccaggctctcgctcggcggtccccgcttccaactcgtccaagcaatctcggtcgtcgtcgtcgtccaactcagcctcgacctcgcgctcgacgtcgacgccggccccgtcagcccgctcgcagaagccacggcacgttgagcgcgagcaggcggcgggaagttcgcggtcgtttgcaaaccgtattgggaaccatcagcctgagacatccactactaacgggaactcgaacgcctccagctctatgacaccacgcccacacaagtccccaccctcagcctcccagccgtcgccgccatccccaaagtctaatcctcagttcccgggcctcctgtttggcgaaatccccccgggccagctcgacgttgccgcgccaggctttggcatcgacaacatccgcccgcgacgaacgtccgaatccaacgtgcacggcctcgcggggcgtcaacgcagcctctccgacctcgaagccgagcccgcgtccccgtccagctggtacagggacgtccacatgtcccgagggagcacgccgaaaacccacgcgcgatctcgcagcgatctctcggcgctgaagcccatcctcacaaccttgcatcccaagtcgcctacgcccgcatcggcgtcggcatcagcaaagacaagggcgaaatcaccaacagtagctgctttcgcggcagccacggtaacggcaacggctgtcgcagcaggggccgcggccgccaccccttcggtctcgcacgcgtcggcagccgccagctccaaactccccgtttccatcagcaggaggcttgcgagcccaaccaactccagtcctacctcttcgcgatcagcctcgccctctgcgcttcgcagactgccagccaatggtcgtagcaatagcagacaggccaaaccgatcgcccccacgaaccgagcgaaaacacccacgcagacgggtcgaaagcagcctccccaggggctcgttacaccgagtaatagcaacaaccggctccaagcgtacgtgtccaccgcaccacccaagctctcaccgccattgaggagctctcgaccgcgacagccagtctccgtagcgacgacagcaagctcccggatgaaagaggcggcggccaaggcgaagccccctgcacggccaaatgcacgggacggcccttcgtcgtccaagacgtacgatccggcgaagcgtaaaatcatagtgggacccatagacttcgagcagcgtcgggaacacattcgactggcatacaccaagaccattcgcgaaagccaggcctttgaggtgcgccagaaagcggtggagaggaggcggaagcagatggaggaggccaaatcggaagccgcagccaccgaagctacagcgacaagtagcacagctacaggcccatctacgtctgcggattcaccgaccacggtgactgctggagatgacgaggcgacgaataaggggccctcggacggccatgcacgagtaattccagaagtaatagcggcagagacagggctggtcgcgagcccctcggcacaggagccggctgtggaactcccagctgatcatggctctcgcgctgctgtggagaaccagggaacccttctagaaatcccgacagatgcagcctcgcacccggcatctggcaagggtgatgattcgccgacgctgggcatccccgggggcttcccagaaccctcaccgtcctccgccgcggccagaacgcagcggcctctgtcgaccatttcaatcatttcagccacgtctgctgttacggagtttgatacagagccccaggcggagctcgccgacctggctatgtccgatcggccgctcagcccccagatcatagtgccgacgagggagcgctctcagtatagaagcccctttgaggacgacgatttcccatcgtcgccgcctcggccgcgtcctgcggcccatccccatcaaatcagccaagatcgccaccatatcgaccagccgttcatccctgacgcttattacgacaatgagcatcgggagcaccccttggagacccatgcccagcaagattaccaaaccattgtgacgattctgccgcagccctcacgcgagccccctgctgccgaggagactgcgcaggcggcatcttttccccgactagacattcaagatgaatctgactgccactcggatctagaaagcgttccggcaatggctcgtcgcatgcgtagtgatgtcgacgatgccgccactgatgcctgcaccgaagagacggacgaccgcgatgggatggaggacgagcggtccccgtaccgctatggcggcaccctctcgtcgaacagggcctctacatgcgcatcatcggacatagaaacgtttgacgaccttttgtacccttcgcatgacgagcagctggatgcgggcccaccaaacagactactggcgccgccctcgatctctcgggccgacagatcgagccaccagaccgcatggacgaatgtctccgtcgaaagcattacgcgctccgaggcttcagactctcctgttctacgagccagctcctggaagatgtccggttcgtccgggcgagacgactcttccctcagaagcctcccctatagcggagtcggcgtccgcccttcagtcgactcgacgcggtcgtcgattcagcttggccagcagctcccggagctagacacgggcgaaggcttctccatcccgtatctgtccgcagaggcaacctccgacctctcctacctctcttcccccgggaaacacgagccagcacctcacccgcgctctggacggaactcggccatcgactcgcaaacgtcgagcgtgttctatgagcagtcccagtatggcagcacgttggtcaactccgatcgcgggagtggagaatatgtctcccaccattcggaaactcctctgtcgatgatggacttgacctcgatggagactatggatcggtactatgacggtcgcacccaggtggatagcgatgccaagtcattcatccaagaatccgaggggctaagcagcgaagagcggcatcgccttatccagaggcgtaacgtcatcaaggagcttgtggataccgaagcaatttttgtaagagacatgaacattgtggaggaaatctacaagggcaccgcagaggcgtgccccaagctggacaccaagattgtcaagctcatcttcaggaacagcgacgagattatcgagtttcacacctcgttcctcgtcctcctcaaagaggcagtggccagcatttacgtaccgaagggcggccggtctctcgtcgcgagagaagactccatctattcggaacaaggccagacttccatcgtcgacctcagcgatgccaaggacagagagacgtcgctcggtccaactttccaagccaacatggagaagatgaagcttgctcacgaagggttcctgcgaaacagcgaccaagcagcaaagaaactgatccagatccagcaggacccgacggtgcagatatggctgaacgagtgtaatgaggtagccaaggacctgacagctgcctgggatctggactcccttctcatcaagccgatgcaacgaatcacaaagtatccgaatctgatcatgacgctccttcagcacacgccccaggaccaccccgatcgggaggccctcgtgatggccaaagaggcgctcgaagaggccattatcgagatcaacaagacgaagaagaactttgagctggtcggacagatcgtcggtagaaaacgtaaggagtccgacgtgaaggccggactcgctcgtgcctttggcaagaaggtggacaagctgcaaggcggaactcggccaccggaggatccagaatatctcaagctggaggaaaggttcagcgacgattacttgcggctacaggtcgtcttgcgcgatgtcgagttctacacccggcaagtctcatcgtatgtgcacgagttcctgcagtacctatcagccatcgagctggtcatgcgtcttcagccgggcagcttccccgagctggagagcaagtgggtgcgcttcaatatctccattcgcgacattgagaaggtagcgctcgagcagcatctgtcacagattcgaaagcatgtcattgagccttttgagcaggtcatcaagtcctacgggaacccctcgctggctatgaagaagagacaaaagcgccgagtcgtgtgggagcgcgcagagcagctgaagaaggcaggcaaaagcgtcgatcccaagcttaaggagctggttgagcagtatgaagctctcaacgatacgctaatcaaggagcttcccaagctttcggcgctgacggagaaggtgggaaacatttgtctcagcaatctcatcaacatccaggccaattggtacttcatttggagggagaagatgagagctgtgctgccggactcgcccacgatgccagacattgaggaaatagtctcgactttccagcgagactttccctatgcgaacgagatgatggccagcatcggcatcatcaacccagcctaccgcggaaggacatcacaatcaacgaaccacggagacgacgccggcctgcccaggactagaggccgaacctcagagtcggtagataggggatggagtcagtctttcaacggcgaaggcgcaccaagcctaccgcctcccgattttggcaaacggcatagcggctcgtttaccctctcccccatcagcgccggcccgtcctcctctggcttcgggacgtcagctcccagtccccaccagtactattatcgtgacttttatgccggcctgtcaagcaaccaggcgggaatgacatccccgagatcggccgaggtaccagcaacctctcgatcgctcggaggcacgcggccgagtacgggcaaaagctacgattcgtcggcaatctcaatgccgagacagagcacggagtcggcgcctcacatccgacgggactcgggcacggcgtactattccagctaccaccagcacgacagccgtagattctcaaatctctttcactctgcccttcctctaccggacggccccgaagagagtcagcggtcctctagagcatcatcgcgggagcgagcacatgcttctgatgggtacaatatcctatggctggcagcgtcgctcttcgagtttaacatctcgacaaccaagcacgaggctggctatccttatctgacgtatcaggccggcgagatattcgatgtaattgccgagaaaggcgagctttggcttgccaagaatcaagacgacccaacggaccaggtgggctggatctggtccaaacactttgcgaaattggccgactcatag

SEQ ID NO: 4 - protein, 1244 aa, Hypocrea jecorina
MDPDPDLHLPARVERASLDGSVFVAHDAHPPARHLPHLHLPQLAHPASNSTSNFTLRSHSHSPAAGDVVLPASTANAHVLAHHAAATLDPDDYYRSYETASSPGGEPQSLPMASPVPPPTPDGAAARHLQPPANRSAGRQGTSNTAVRSASSPLDRRPAAAAPSSRSPSASAPSVRDLKKRFDQNSGPNSIPRAPPRQAAPVARVSKQESSKPKPKPKPSLSAPSSTHPGSRSAVPASNSSKQSRSSSSSNSASTSRSTSTPAPSARSQKPRHVEREQAAGSSRSFANRIGNHQPETSTTNGNSNASSSMTPRPHKSPPSASQPSPPSPKSNPQFPGLLFGEIPPGQLDVAAPGFGIDNIRPRRTSESNVHGLAGRQRSLSDLEAEPASPSSWYRDVHMSRGSTPKTHARSRSDLSALKPILTTLHPKSPTPASASASAKTRAKSPTVAAFAAATVTATAVAAGAAAATPSVSHASAAASSKLPVSISRRLASPTNSSPTSSRSASPSALRRLPANGRSNSRQAKPIAPTNRAKTPTQTGRKQPPQGLVTPSNSNNRLQAYVSTAPPKLSPPLRSSRPRQPVSVATTASSRMKEAAAKAKPPARPNARDGPSSSKTYDPAKRKIIVGPIDFEQRREHIRLAYTKTIRESQAFEVRQKAVERRRKQMEEAKSEAAATEATATSSTATGPSTSADSPTTVTAGDDEATNKGPSDGHARVIPEVIAAETGLVASPSAQEPAVELPADHGSRAAVENQGTLLEIPTDAASHPASGKGDDSPTLGIPGGFPEPSPSSAAARTQRPLSTISIISATSAVTEFDTEPQAELADLAMSDRPLSPQIIVPTRERSQYRSPFEDDDFPSSPPRPRPAAHPHQISQDRHHIDQPFIPDAYYDNEHREHPLETHAQQDYQTIVTILPQPSREPPAAEETAQAASFPRLDIQDESDCHSDLESVPAMARRMRSDVDDAATDACTEETDDRDGMEDERSPYRYGGTLSSNRASTCASSDIETFDDLLYPSHDEQLDAGPPNRLLAPPSISRADRSSHQTAWTNVSVESITRSEASDSPVLRASSWKMSGSSGRDDSSLRSLPYSGVGVRPSVDSTRSSIQLGQQLPELDTGEGFSIPYLSAEATSDLSYLSSPGKHEPAPHPRSGRNSAIDSQTSSVFYEQSQYGSTLVNSDRGSGEYVSHHSETPLSMMDLTSMETMDRYYDGRTQVDSDAKSFIQESEGLSSEERHRLIQRRNVIK

SEQ ID NO: 5 - protein, 213 aa, Hypocrea jecorina
VIEELVDTEAIFVRDMNIVEEIYKGTAEACPKLDTKIVKLIFRNSDEIIEFHTSFLVLLKEAVASIYVPKGGRSLVAREDSIYSEQGQTSIVDLSDAKDRETSLGPTFQANMEKMKLAHEGFLRNSDQAAKKLIQIQQDPTVQIWLNECNEVAKDLTAAWDLDSLLIKPMQRITKYPNLIMTLLQHTPQDHPDREALVMAKEALEEAIIEINK

SEQ ID NO: 6 - protein, 102 aa, Hypocrea jecorina
ALEQHLSQIRKHVIEPFEQVIKSYGNPSLAMKKRQKRRVVWERAEQLKKAGKSVDPKLKELVEQYEALNDTLIKELPKLSALTEKVGNICLSNLINIQANWY

SEQ ID NO: 7 - protein, 72 aa, Hypocrea jecorina
GYNILWLAASLFEFNISTTKHEAGYPYLTYQAGEIFDVIAEKGELWLAKNQDDPTDQVGWIWSKHFAKLADS

SEQ ID NO: 8 - RNA, 20 nt, Artificial Sequence
  synthetic
cauucauccaagaauccgag

SEQ ID NO: 9 - DNA, 21 bp, Artificial Sequence
  primer
cctaactaacgtctgacatcg

SEQ ID NO: 10 - DNA, 23 bp, Artificial Sequence
  primer
cgtaccatttgactgatacgatg

SEQ ID NO: 11 - DNA, 19 bp, Artificial Sequence
  primer
gagctagacacgggcgaag

SEQ ID NO: 12 - DNA, 20 bp, Artificial Sequence
  primer
aggaacccttcgtgagcaag

SEQ ID NO: 13 - RNA, 20 nt, Artificial Sequence
  synthetic
auuguggaggaaaucuacaa

SEQ ID NO: 14 - DNA, 1026 bp, Artificial Sequence
  synthetic
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcgcctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatag

SEQ ID NO: 15 - DNA, 801 bp, Artificial Sequence
  synthetic
catcagcgttggctttccgtctccattggctcttggcaattcggtcagcggggctgactgcctcaggtggggcagtgctagtgtgtgtaccgacccgcaggattggtgctttgcccagagctctacagaatagcgcgcgcatccatatgttagttctgcaattttcttgtatcggtgctgtgactcatacttccccctttggctggccttgcggcaaccaataagaacgcacagtgaaatcttgcgggtggggagtggatccatggcgcctgcattggcttggggacgcgcactgtcgcacacttccatctgacctttcagaagggtttcgtggtgggcaaggaccaaccggttgcgcggccgtgcgtgggtgcctcgcccggcactgccagggccactgcagtggcagtttgctgcctgatacaaaatccttccctccgcccagttttccctctttgaccttcctttcttcttctctgcaaccaaatccaccctatcaaaccaaaacagtatctcgaccgaggtatcaacctgaatcagcaacatcgtagccagcatttgtctccgtctctgcagaaccagcgagttgcaaacattatccaggcaacagggcaccaactcacttcttcggctttcaccaatcggtacagctcttctcagaactcgcgtccgcaacagttctacgcttcctcagcaccttcttcagcttcaatcctgaacactcagaaccgcgcacagcagcgccctcctgttcccttgtttcccaaaagtaccggtagtatttcgcacggaaagcagggc

SEQ ID NO: 16 - DNA, 741 bp, Artificial Sequence
  synthetic
agtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcccctgggttcgcaaagataattgcactgtttcttccttgaactctcaagcctacaggacacacattcatcgtaggtataaacctcgaaaatcattcctactaagatgggtatacaatagtaaccatggttgcctagtgaatgctccgtaacacccaatacgccggccgaaacttttttacaactctcctatgagtcgtttacccagaatgcacaggtacacttgtttagaggtaatccttctttctag

SEQ ID NO: 17 - DNA, 19 bp, Artificial Sequence
  primer
tggctttccgtctccattg

SEQ ID NO: 18 - DNA, 21 bp, Artificial Sequence
  primer
agtgtacctgtgcattctggg

SEQ ID NO: 19 - DNA, 1957 bp, Artificial Sequence
  synthetic
attaaggaggggacgatacacgcaccatggaccccagtggggaagcaagagcgggaaggagagctgcgagacgtacagtgtgaattgcttcacgtctaaagtatcaaggtatggagctggatggatgggctaaggtagtagacggcttccctcagcaactgagcttagttatgatgctataacgtccactttgatctgcgaaattgggcaaaaaaaaaaaaaaaaaaaaaaaaaaaaaaaaaaaaaaaaagaatctattgcattaatggacatatataaaggtcaaaaggccgcagaattgacgtcgtagtctagccaggtagttgatgccggatctatgctcttcaccgttcagagacgaaacttttacttcattagggggagtccaacttttcattctcactctgtgagaagctgtgtcacaatacttcagaggcattaaagctacctcagctctttaaacatgtcaattctatcaaaattaggtatgtgattcaagtagtgaacaatatgtggccgttactcgagtttataggtgacaacatgctctcaaagcgctcatggctggcacaagcctggaaagaaccaacacaaagcatactgcagcaaatcagctgaattcgtcaccaattaagtgaacatcaacctgaaggcagagtatgaggccagaagcacatctggatcgcagatcatggattgcccctcttgttgaagatgagaatctagaaagatggcggggtatgagataagagcgatgggggggcacatcatcttccaagacaaacaacctttgcagagtcaggcaatttttcgtataagagcaggaggagggagtccagtcatttcatcagcggtaaaatcactctagacaatcttcaagatgagttctgccttgggtgacttatagccatcatcatacctagacagaagcttgtgggatactaagaccaacgtacaagctcgcactgtacgctttgacttccatgtgaaaactcgatacggcgcgcctctaaattttatagctcaaccactccaatccaacctctgcatccctctcactcgtcctgatctactgttcaaatcagagaataaggacactatccaaatccaacagaatggctaccacctcccagctgcctgcctacaagcaggacttcctcaaatccgccatcgacggcggcgtcctcaagtttggcagcttcgagctcaagtccaagcggatatccccctacttcttcaacgcgggcgaattccacacggcgcgcctcgccggcgccatcgcctccgcctttgcaaagaccatcatcgaggcccaggagaaggccggcctagagttcgacatcgtcttcggcccggcctacaagggcatcccgctgtgctccgccatcaccatcaagctcggcgagctggcgccccagaacctggaccgcgtctcctactcgtttgaccgcaaggaggccaaggaccacggcgagggcggcaacatcgtcggcgcttcgctcaagggcaagagggtcctgattgtcgacgacgtcatcaccgccggcaccgccaagagggacgccattgagaagatcaccaaggagggcggcatcgtcgccggcatcgtcgtggccctggaccgcatggagaagctccccgctgcggatggcgacgactccaagcctggaccgagtgccattggcgagctgaggaaggagtacggcatccccatctttgccatcctcactctggatgacattatcgatggcatgaagggctttgctacccctgaggatatcaagaacacggaggattaccgtgccaagtacaaggcgactgactgattgaggcgttcaatgtcagaagggagagtaagactgaaaaggtggaaagaagaggcaaattgttgttattattattattctatctcgaatcttctagatcttgtcgtaaataaacaagcgtaactagctag

(19) United States
(12) Patent Application Publication
(10) Pub. No.: US 2024/0102070 A1
(43) Pub. Date: Mar. 28, 2024

(54) FUNGAL STRAINS COMPRISING ENHANCED PROTEIN PRODUCTIVITY PHENOTYPES AND METHODS THEREOF

(71) Applicant: DANISCO US INC., Palo Alto, CA (US)

(72) Inventors: Elizabeth A. Bodie, Belmont, CA (US); Zhongqiang Chen, Wilmington, DE (US); Chuanbin Liu, Menlo Park, CA (US); Michael Ward, San Francisco, CA (US)

(21) Appl. No.: 17/766,902

(22) PCT Filed: Nov. 6, 2020
(86) PCT No.: PCT/US2020/059379

§ 371 Date: Apr. 6, 2022

Related U.S. Application Data
(60) Provisional application No. 62/932,525, filed on Nov. 8, 2019.

(51) Int. Cl. C12P 21/02; C12N 9/22; C12N 9/24; C12N 15/11; C12N 15/90

(52) U.S. Cl. CPC C12P 21/02; C12N 9/22; C12N 9/2402; C12N 15/11; C12N 15/905; C12Y 302/01; C12N 2310/20; C12N 2800/80

(57) ABSTRACT

The present disclosure is generally related to modified filamentous fungal strains (cells) comprising enhanced protein productivity phenotypes, wherein such modified strains are particularly well-suited for growth in submerged cultures (e.g., large-scale production of proteins for industrial/commercial applications). Thus, certain embodiments of the disclosure are related to such variant (modified) strains of filamentous fungus derived (obtained) from parental strains comprising a gene encoding a native GEF1 protein, wherein the variant strain comprises a genetic modification which disrupts or deletes a gene encoding a native GEF1 protein, wherein the variant strain comprises an increased protein productivity phenotype (i.e., relative to the parental strain) when grown/cultivated/fermented under the same conditions.

### TECHNICAL FIELD

The present disclosure is generally related to the fields of biology, molecular biology, filamentous fungi, yeast, fermentation, genetics, industrial protein production and the like. More particularly, the present strains and methods of the disclosure relate to genetic modifications in filamentous fungi that give rise to variant (modified) strains having altered phenotypes, wherein such modified strains are particularly well-suited for growth in submerged cultures (e.g., large-scale production of proteins for industrial/commercial applications).

### CROSS REFERENCE TO RELATED APPLICATIONS

This application claims benefit to U.S. Provisional Patent Application No. 62/932,525, filed Nov. 8, 2019, which is incorporated herein by referenced in its entirety.

### REFERENCE TO A SEQUENCE LISTING

The contents of the electronic submission of the text file Sequence Listing, named “NB41383-WO-PCT_SequenceListing.txt” was created on Oct. 27, 2020 and is 55 KB in size, which is hereby incorporated by reference in its entirety.

### BACKGROUND

Filamentous fungi (e.g., Aspergillus sp., Penicillium sp., Talaromyces sp., Fusarium sp., Myceliophthora sp., Neurospora sp., Candida sp., Trichoderma sp., and the like) are capable of expressing native and heterologous proteins to high levels, making them well-suited for the large-scale production of proteins (e.g., enzymes, antibodies, peptides, etc.) and/or metabolites for industrial and/or commercial applications such as pharmaceutical applications, animal health applications, food applications, beverage applications, laundry and textile applications, and the like. Filamentous fungi are typically grown in mycelial submerged cultures in bioreactors (fermentors), which bioreactors are adapted to introduce and distribute oxygen and nutrients into the culture medium (i.e., culture broth). For example, the filamentous fungus Trichoderma reesei (T. reesei; an anamorph of the fungus Hypocrea jecorina) is known to be an efficient producer of cellulase enzymes.

As such, filamentous fungi have been utilized for their ability to produce proteins (e.g., enzymes), which proteins are valuable in the production of commodities such as cellulosic (derived) ethanol, textile processing, grain processing, detergents, fibers/pulp/paper, food additives, feed additives and the like. For example, recombinant gene expression in such fungal host strains is a common method for the production of proteins (i.e., for industrial and commercial purposes) and as such, protein productivity improvements of a fungal host strain are an important economic factor of protein production costs. Thus, as appreciated by one of skill in the art, such novel compositions and methods for enhancing protein production in filamentous fungal strains are of significant commercial interest.

### SUMMARY

The present disclosure is generally related to modified filamentous fungal strains (cells) comprising enhanced protein productivity phenotypes, wherein such modified strains are particularly well-suited for growth in submerged cultures (e.g., large-scale production of proteins for industrial/commercial applications). Thus, certain embodiments of the disclosure are related to such variant (modified) strains of filamentous fungus derived (obtained) from parental strains comprising a gene encoding a native GEF1 protein, wherein the variant strain comprises a genetic modification which disrupts or deletes the gene encoding the native GEF1 protein, wherein the variant strain comprises an increased protein productivity phenotype (i.e., relative to the parental strain) when grown/cultivated/fermented under the same conditions.

### BRIEF DESCRIPTION OF THE BIOLOGICAL SEQUENCES

SEQ ID NO: 1 is a T. reesei polynucleotide sequence encoding a native GEF1 protein comprising SEQ ID NO: 2.
SEQ ID NO: 2 is the amino acid sequence of the native full length GEF1 protein encoded by the polynucleotide of SEQ ID NO: 1.
SEQ ID NO: 3 is an open reading frame (ORF) encoding the native full length GEF1 protein of SEQ ID NO: 2.
SEQ ID NO: 4 is a C-terminally truncated GEF1 variant protein comprising amino acid positions 1-1,244 of SEQ ID NO: 2.
SEQ ID NO: 5 is the amino acid sequence of the GEF1 domain present in SEQ ID NO: 2.
SEQ ID NO: 6 is the amino acid sequence of the BAR domain present in SEQ ID NO: 2.
SEQ ID NO: 7 is the amino acid sequence of a conserved, unknown domain present in SEQ ID NO: 2.
SEQ ID NO: 8 is a target site 3 (TS3) RNA sequence within the T. reesei GEF1 gene.
SEQ ID NO: 9 is the nucleic acid sequence of primer AL950.
SEQ ID NO: 10 is the nucleic acid sequence of primer AL952.
SEQ ID NO: 11 is the nucleic acid sequence of primer RhoF1.
SEQ ID NO: 12 is the nucleic acid sequence of primer RhoR1.
SEQ ID NO: 13 is a target site 4 (TS4) RNA sequence within the T. reesei GEF1 gene.
SEQ ID NO: 14 is the nucleic acid sequence of a hygromycin phosphotransferase marker gene.
SEQ ID NO: 15 is a Neurospora crassa cpcl promoter nucleic acid sequence.
SEQ ID NO: 16 is an Aspergillus nidulans trpC terminator nucleic acid sequence.
SEQ ID NO: 17 is the nucleic acid sequence of primer HygF1.
SEQ ID NO: 18 is the nucleic acid sequence of primer HygR1.
SEQ ID NO: 19 is the nucleic acid sequence of a pyr2 marker gene.

### BRIEF DESCRIPTION OF THE DRAWINGS

FIG. 1 presents a polynucleotide sequence (SEQ ID NO: 1; FIG. 1A-FIG. 1B) encoding a native Trichoderma sp. GEF1 protein homologue comprising the amino acid sequence of SEQ ID NO: 2. As shown in FIG. 1A and FIG. 1B, the DNA sequence encoding the GEF1 protein (SEQ ID NO: 2) comprises two (2) introns, presented in lower case (nucleotide) letters and three (3) exons, presented in upper case (nucleotide) letters, wherein the Cas9 target site 3 (TS3) nucleotides are presented as double underlined upper case letters, the Cas9 target site 4 (TS4) nucleotides are presented as single underlined upper case letters and the bold upper case codon GAG of the wild-type sequence (SEQ ID NO: 1), which codon was mutated to a premature (TAG) stop codon in mutant strain, thereby encoding a C-terminally truncated GEF1 protein (FIG. 1B).

FIG. 2 shows the open reading frame (ORF) sequence (SEQ ID NO: 3; FIG. 2A-FIG. 2B) encoding the native GEF1 protein of SEQ ID NO: 2.

FIG. 3 shows the amino acid sequences of a native GEF1 protein (SEQ ID NO: 2; FIG. 3A) and a variant (C-terminally truncated) GEF1 protein (SEQ ID NO: 4; FIG. 3B). Also shown in FIG. 3 are the GEF1 domain (FIG. 3C; SEQ ID NO: 5), the BAR domain (FIG. 3D; SEQ ID NO: 6) and an unknown domain (FIG. 3E; SEQ ID NO: 7) of the native GEF1 protein of SEQ ID NO: 2. For example, the amino acid sequence of the native GEF1 protein (SEQ ID NO: 2; FIG. 3A) comprises a Pfam predicted GEF1 domain (FIG. 3A, light grey amino acid shading; PF00621) and a BAR domain (FIG. 3A, bold amino acids; PF03114). As shown in FIG. 3B, the variant GEF1 protein (SEQ ID NO: 4) is C-terminally truncated, wherein the truncation occurs near the N-terminus of the GEF1 domain (e.g., residues “VIK”).

### DETAILED DESCRIPTION

As set forth and described herein, the present disclosure is generally related to genetically modified filamentous fungal strains (cells) and their use in the production industrial proteins. More particularly, the present strains and methods of the disclosure relate to genetic modifications in filamentous fungi that give rise to variant strains having altered phenotypes, wherein such variant strains are particularly well-suited for growth in submerged cultures (e.g., large-scale production of proteins for industrial/commercial applications). Thus, as described herein, certain embodiments of the disclosure are related to genetically modified strains of filamentous fungus and methods thereof, wherein such modified strains comprise enhanced protein productivity phenotypes, such as improved volumetric efficiencies, higher specific productivities, improved yield on carbon sources, reduced bioreactor (fermentor) operating costs and the like.

### I. DEFINITIONS

Prior to describing the present strains and methods in detail, the following terms are defined for clarity. Terms not defined should be accorded their ordinary meanings as used in the relevant art. Unless defined otherwise, all technical and scientific terms used herein have the same meaning as commonly understood by one of ordinary skill in the art to which the present compositions and methods apply.

All publications and patents cited in this specification are herein incorporated by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. For example, in connection with a numerical value, the term “about” refers to a range of ⁻10% to ⁺10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

In accordance with this Detailed Description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely”, “only”, “excluding”, “not including” and the like in connection with the recitation of claim elements, or use of a “negative” limitation.

It is further noted that the term “comprising”, as used herein, means “including, but not limited to”, the component(s) after the term “comprising”. The component(s) after the term “comprising” are required or mandatory, but the composition comprising the component(s) may further include other non-mandatory or optional component(s).

It is also noted that the term “consisting of,” as used herein, means “including and limited to”, the component(s) after the term “consisting of”. The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

Filamentous fungus cells for manipulation, construction and use as described herein are generally from the phylum Ascomycota, subphylum Pezizomycotina, particularly fungi that have a vegetative hyphae state. Such organisms include filamentous fungus cells used for the production of commercially important industrial and pharmaceutical proteins, including, but not limited to Trichoderma sp., Aspergillus sp., Fusarium sp., Penicillium sp., Chrysosporium sp., Cephalosporium sp., Talaromyces sp., Geosmithia sp., Neurospora sp., Myceliophthora sp. and the like.

For example, in certain embodiments, filamentous fungus cells and strains thereof include, but are not limited to Trichoderma reesei (previously classified as Trichoderma longibrachiatum and Hypocrea jecorina), Aspergillus niger, Aspergillus fumigatus, Aspergillus itaconicus, Aspergillus oryzae, Aspergillus nidulans, Aspergillus terreus, Aspergillus sojae, Aspergillus japonicus, Neurospora crassa, Penicillium funiculosum, Penicillium chrysogenum, Talaromyces (Geosmithia) emersonii, Fusarium venenatum, Myceliophthora thermophila, Chrysosporium lucknowense (C1) and the like.

As used herein, terms and phrases such as “filamentous fungus strain(s)”, “filamentous fungal strain(s)”, “fungus strain(s)”, “fungal strains(s)”, “filamentous fungus cell(s)”, “filamentous fungal cell(s)”, “fungus cell(s)”, “fungal cell(s)” and the like may be used interchangeably for convenience of description, and are not intend to limit the scope of the disclosure.

As used herein, “yeast” and/or “yeast strains” include, but are not limited to, Saccharomyces sp. (e.g., S. cerevisiae, S. pastorianus, S. carlsbergensis), Schizosaccharomyces sp. (e.g., S. pombe), Yarrowia sp. (e.g., Y. lipolytica) and the like.

As used herein, the term “gene” is synonymous with the term “allele” in referring to a nucleic acid (polynucleotide) that encodes and directs the expression of a protein or RNA. Vegetative forms of filamentous fungi are generally haploid, therefore a single copy of a specified gene (i.e., a single allele) is sufficient to confer a specified phenotype.

As used herein, phrases such as a “parental cell”, a “parental fungal cell”, a “parental strain”, a “parental fungal strain”, a “parental strain of filamentous fungus cells”, “reference strain” and the like may be used interchangeably, and refer to “unmodified” parental filamentous fungal cells. For example, a “parental strain of filamentous fungus cells” refers to any cell or strain of filamentous fungi in which the genome of the “parental” cell is modified (e.g., via a genetic modification introduced into the parental cell) to generate a variant (daughter) strain of filamentous fungus cells, such that “parental” and “daughter” cells differ.

As used herein, phrases such as a “variant cell”, a “daughter cell”, a “variant strain”, a “daughter strain”, a “variant or daughter fungal strain”, a “variant or daughter strain of filamentous fungus cells” and the like may be used interchangeably, and refer to the modified strains of filamentous fungus cells that are derived (i.e., obtained from or obtainable from) from a parental (or reference) strain of filamentous fungus cells, wherein the variant strain comprises a genetic modification which is not present in the parental strain, such that, by comparison, phenotypic differences between the “parental” and “variant” strains can be attributed to the genetic modification. In other terms, parental and variant strains are otherwise isogenic except for the genetic modification(s) “introduced” into the variant strain.

Thus, parental and variant “strains” can be described as having certain characteristics, such as genetic modifications, expression phenotypes, morphology phenotypes and the like; however, the skilled person will appreciate that it is technically the “cells” of the parental or variant strain that have such characteristics, and the “strains” are referred to for convenience.

As used herein, the terms “wild-type” and “native” are used interchangeably and refer to genes, proteins, protein mixes or strains, as found in nature.

As used herein, an “endogenous filamentous fungal gene” encoding a protein of interest includes, but is not limited to, endogenous genes encoding glycoside hydrolase (GH) family enzymes (e.g., such as EC Nos. 3.2.1.1-3.2.1.206), endogenous genes encoding proteases, endogenous genes encoding esterases, endogenous genes encoding lipases and the like, as known and understood by one skilled in the art.

As used herein, the phrases “lignocellulosic degrading enzymes”, “cellulase enzymes”, and “cellulases” are used interchangeably, and include glycoside hydrolase (GH) enzymes such as cellobiohydrolases, xylanases, endoglucanases, and β-glucosidases, that hydrolyze the β-(1,4)-linked glycosidic bonds of cellulose (hemi-cellulose) to produce glucose.

In certain embodiments, cellobiohydrolases include enzymes classified under Enzyme Commission No. (EC 3.2.1.91), endoglucanases include enzymes classified under EC 3.2.1.4, endo-β-1,4-xylanases include enzymes classified under EC 3.2.1.8, β-xylosidases include enzymes classified under EC 3.2.1.37, and β-glucosidases include enzymes classified under EC 3.2.1.21.

As used herein, “endoglucanase” proteins may be abbreviated as “EG”, “cellobiohydrolase” proteins may be abbreviated “CBH”, “3-glucosidase” proteins may be abbreviated “BG” and “xylanase” proteins may be abbreviated “XYL”. Thus, as used herein, a gene (or ORF) encoding a EG protein may be abbreviated “eg”, a gene (or ORF) encoding a CBH protein may be abbreviated “cbh”, a gene (or ORF) encoding a BG protein may be abbreviated “bg”, and a gene (or ORF) encoding a XYL protein may be abbreviated “xyl”.

In certain embodiments, filamentous fungus cells for manipulation, construction and use as described herein are generally from the subphylum Pezizomycotina, particularly fungi that have a vegetative hyphae state and comprise a GEF1 gene or a homologue thereof.

As used herein, a “gene or polynucleotide encoding a native GEF1 protein” comprises sequence homology to SEQ ID NO: 1. In certain embodiments, a gene or polynucleotide encoding a native GEF1 protein comprises about 70% to 100% sequence identity to SEQ ID NO: 1 or SEQ ID NO: 3. In certain other embodiments, a gene or polynucleotide encoding a native GEF1 protein comprises about 70% to 100% sequence identity to SEQ ID NO: 1 or SEQ ID NO: 3, and encodes a GEF1 domain comprising at least 90% sequence identity to SEQ ID NO: 5. In certain other embodiments, a gene or polynucleotide encoding a native GEF1 protein hybridizes with a nucleic acid sequence of SEQ ID NO: 1 or SEQ ID NO: 3, under medium to stringent hybridization conditions.

In certain embodiments, an upstream (5′) cellulase gene promoter sequence includes, but is not limited to, a cellobiohydrolase (cbh) gene promoter sequence, an endoglucanase (eg) gene promoter sequence, a β-glucosidase (bg) gene promoter sequence, a xylanase (xyl) gene promoter sequence, and the like.

As used herein, an “open reading frame (ORF) nucleic acid sequence encoding a native Trichoderma sp. GEF1 protein” comprises sequence homology to the ORF nucleic acid sequence of SEQ ID NO: 3. In certain other embodiments, an ORF nucleic acid sequence encodes a native Trichoderma sp. GEF1 protein comprising about 70% to 100% sequence identity to SEQ ID NO: 2. In other embodiments, an ORF nucleic acid sequence encodes a native Trichoderma sp. GEF1 protein comprising about 70% to 100% sequence identity to SEQ ID NO: 2 and comprises a GEF1 domain having at least 90% sequence identity to SEQ ID NO: 5. In certain other embodiments, an ORF encoding a native GEF1 protein hybridizes with a nucleic acid sequence of SEQ ID NO: 1 or SEQ ID NO: 3 under medium to stringent hybridization conditions.

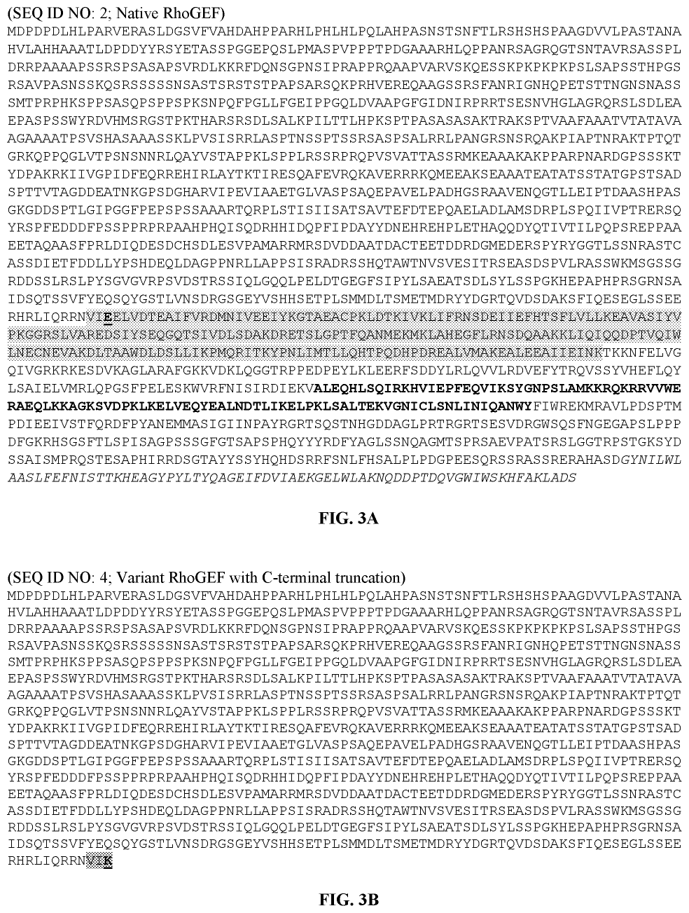

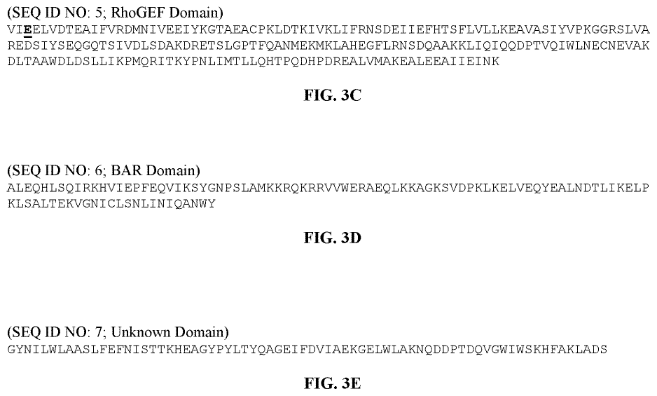

As used herein, the “position” of an amino acid residue in a “given amino acid sequence” is numbered herein using the amino acid residue numbering (positions) of the native Trichoderma sp. GEF1 protein of SEQ ID NO: 2. For example, FIG. 3A presents the amino acid sequence of a native Trichoderma sp. GEF1 protein (SEQ ID NO: 2). Thus, phrases such as “comprises a glutamic acid (E; Glu) residue at a sequence position corresponding to position 1,244 of SEQ ID NO: 2”, or “comprises a GEF1 domain at a sequence position corresponding to positions 1,242-1,454 of SEQ ID NO: 2”, the native (Trichoderma sp.) GEF1 protein's amino acid sequence (SEQ ID NO: 2) serves as a reference (parent) protein sequence. For example, as shown in FIG. 3A, a given amino acid sequence described herein can be aligned with the native Trichoderma sp. GEF1 protein amino acid sequence (SEQ ID NO: 2), using alignment algorithms described herein (and/or alignment algorithms known by one skilled in the art,) and an amino acid residue in the given amino acid sequence that aligns (preferably, optimally aligns) with an amino acid residue in the native sequence can be conveniently numbered by reference to the corresponding amino acid residue in the GEF1 sequence.

Likewise, to establish sequence homology or sequence identity to the primary (10) sequence of the Trichoderma sp. GEF1 protein (SEQ ID NO: 2), one skilled in the art may readily compare the primary sequence of SEQ ID NO: 2 with one or more candidate GEF1 protein homologue/orthologue sequences using sequence alignment algorithms, software and methods thereof know to one skilled in the art. Thus, after aligning the conserved residues, allowing for necessary insertions and deletions in order to maintain alignment (i.e., avoiding the elimination of conserved residues through arbitrary deletion and insertion), the residues equivalent to particular amino acids in the primary sequence of a candidate filamentous fungus GEF1 protein are defined. Alignment of conserved residues preferably should conserve 100% of such residues. However, alignment of greater than 98%, 95%, 90%, 85%, 80%, 75% 70%, 50% or at least 45% of conserved residues is also adequate to define equivalent residues.

As used herein, a Trichoderma strain named “T4” was serially propagated under selective conditions to identify and isolate mutant strains thereof capable of high temperature protein production, without adversely affecting specific productivity (Qp).

As used herein, a mutant Trichoderma strain named “T4-E1” (i.e., derived from parental ‘T4’ strain) was identified under selective temperatures and isolated, wherein T4-E1 mutant strain has a similar specific productivity (Qp) at 28° C. relative to the Qp of the T4 strain at 25° C.

As used herein, a variant Trichoderma strain named “T4-ΔRho #22D” was derived from the T4 parental strain, wherein the variant T4-ΔRho #22D strain comprises a pyr2 marker gene introduced at the Cas9 cleavage site (TS3) thereby disrupting the GEF1 coding sequence.

As used herein, a Trichoderma strain named “t-ATV84” comprises 3 copies of a S. coccosporum gene encoding a DXST endoglucanase and comprises deletions (or disruptions) of native cellulase genes encoding the CBHI, CBHII, EGI and EGII proteins.

As used herein, a Trichoderma strain named “t-AWC88” was derived from the t-ATV84 strain, and further comprises an introduced disruption of the GEF1 gene.

As used herein, the Staphylotrichum coccosporum endoglucanase “STCE1” has been described in U.S. Pat. No. 7,595,182 (incorporated herein by reference in its entirety).

As used herein, the “S. coccosporum STCE1 endoglucanase” may also be referred to as the “S. coccosporum DXST endoglucanase”, and as such, the terms STCE1 and DXST are interchangeable. In certain embodiments, a polynucleotide sequence encoding a DXST protein is placed under the control of a promoter such as cbhI.

As used herein, the phrase “elevated fermentation (cultivation) temperatures” is a fermentation temperature greater than 25° C. In certain embodiments, an elevated fermentation temperature is at least about 25.5° C. to about 28° C. In certain embodiments, an elevated fermentation temperature is at least about 25.1° C. to 25.9° C.

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the term “derivative polypeptide/protein” refers to a protein which is derived or derivable from a protein by addition of one or more amino acids to either or both the N- and C-terminal end(s), substitution of one or more amino acids at one or a number of different sites in the amino acid sequence, deletion of one or more amino acids at either or both ends of the protein or at one or more sites in the amino acid sequence, and/or insertion of one or more amino acids at one or more sites in the amino acid sequence. The preparation of a protein derivative can be achieved by modifying a DNA sequence which encodes for the native protein, transformation of that DNA sequence into a suitable host, and expression of the modified DNA sequence to form the derivative protein.

Related (and derivative) proteins include “variant proteins”. Variant proteins differ from a reference/parental protein (e.g., a wild-type protein) by substitutions, deletions, and/or insertions at a small number of amino acid residues. The number of differing amino acid residues between the variant and parental protein can be one or more, for example, 1, 2, 3, 4, 5, 6, 7, 8, 9, 10, 15, 20, 30, 40, 50, or more amino acid residues. Variant proteins can share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a reference protein. A variant protein can also differ from a reference protein in selected motifs, domains, epitopes, conserved regions, and the like.

As used herein, the term “analogous sequence” refers to a sequence within a protein that provides similar function, tertiary structure, and/or conserved residues as the protein of interest (i.e., typically the original protein of interest). For example, in epitope regions that contain an α-helix or a β-sheet structure, the replacement amino acids in the analogous sequence preferably maintain the same specific structure.

The term also refers to nucleotide sequences, as well as amino acid sequences. In some embodiments, analogous sequences are developed such that the replacement of amino acids result in a variant enzyme showing a similar or improved function. In some embodiments, the tertiary structure and/or conserved residues of the amino acids in the protein of interest are located at or near the segment or fragment of interest. Thus, where the segment or fragment of interest contains, for example, an α-helix or a β-sheet structure, the replacement amino acids preferably maintain that specific structure.

As used herein, the term “homologous protein” refers to a protein that has similar activity and/or structure to a reference protein. It is not intended that homologues necessarily be evolutionarily related. Thus, it is intended that the term encompass the same, similar, or corresponding protein(s) (i.e., in terms of structure and function) obtained from different organisms. In some embodiments, it is desirable to identify a homologue that has a quaternary, tertiary and/or primary structure similar to the reference protein.

The degree of homology between sequences can be determined using any suitable method known in the art (see, e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, WI); and Devereux et al., 1984).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (1987). The method is similar to that described by Higgins and Sharp (1989). Useful PILEUP parameters including a default gap weight of 3.00, a default gap length weight of 0.10, and weighted end gaps. Another example of a useful algorithm is the BLAST algorithm, described by Altschul et al., 1990 and Karlin et al., 1993. One particularly useful BLAST program is the WU-BLAST-2 program (see, e.g., Altschul et al., 1996). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff, 1989) alignments (B) of 50, expectation (E) of 10, M′5, N′-4, and a comparison of both strands.

As used herein, the phrases “substantially similar” and “substantially identical”, in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity can be determined using known programs such as BLAST, ALIGN, and CLUSTAL using standard parameters. (See, e.g., Altschul, et al., 1990; Henikoff et al., 1989; Karlin et al., 1993; and Higgins et al., 1988). Software for performing BLAST analyses is publicly available through the National Center for Biotechnology Information. Also, databases can be searched using FASTA (Pearson et al., 1988). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand.

As used herein, the term “expression” refers to the transcription and stable accumulation of sense (mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to transcription, post-transcriptional modification, translation, post-translational modification, secretion and the like.

As used herein, the combined term “expresses/produces”, as used in phrases such as a “variant strain of filamentous fungus cells expresses/produces an ‘increased’ amount of a protein of interest (POI)” (i.e., relative to the parental cell), the term “expresses/produces” is meant to include any steps involved in the expression and production of a protein in such variant filamentous fungus strains of the disclosure.

In certain embodiments, a gene, polynucleotide or nucleic acid sequence encoding a native GEF1 protein comprising “sequence homology” refers to DNA or RNA (nucleic acid) sequences that have de minimus sequence variations from the corresponding nucleic acid sequences (to which comparison is made) and retain substantially the same biological functions as the corresponding nucleic acid sequences (to which comparison is made). For example, in certain embodiments, a nucleic acid sequence comprising substantial sequence homology to a gene, polynucleotide, or nucleic acid encoding a native GEF1 protein is assessed by identifying the encoded gene product (native GEF1 protein), as described herein.

In certain other embodiments, a gene, polynucleotide, or nucleic acid sequence comprising sequence homology to a gene, polynucleotide, or nucleic acid encoding a native GEF1 protein is determined/identified using nucleic acid hybridization methods. For example, in certain embodiments, a DNA/RNA sequence comprising substantial sequence homology to a gene encoding a native GEF1 protein (e.g., SEQ ID NO: 2) is identified by the ability of such DNA/RNA sequence to hybridize with a specified nucleic acid sequence of the disclosure, under stringent conditions.

As used herein, “hybridizes under stringent conditions” is intended to describe conditions for hybridization and washing under which nucleotide sequences that are significantly identical or homologous to each other remain hybridized to each other. Such stringent conditions are well known to those skilled in the art (see, e.g., Ausubel et al., 1995; Sambrook et al., 1989). For example, in certain embodiments, a non-limiting example of stringent hybridization conditions includes hybridization in 4× sodium chlorine/sodium citrate (SSC), at about 65-70° C. (or hybridization in 4×SSC plus 50% formamide at about 42-50° C.), followed by one or more washes in 1×SSC, at about 65-70° C. Likewise, anon-limiting example of highly stringent hybridization conditions includes hybridization in 1×SSC, at about 65-70° C. (or hybridization in 4×SSC plus 50% formamide at about 42-50° C.), followed by one or more washes in 0.3×SSC, at about 65-70° C. Thus, highly stringent hybridization conditions include hybridization in 4×SSC, at about 50-60° C. (or alternatively hybridization in 6×SSC plus 50% formamide at about 40-45° C.), followed by one or more washes in 2×SSC, at about 50-60° C. Ranges intermediate to the above-recited values, e.g., at 65-70° C. or at 42-50° C. are also intended to be encompassed by the present disclosure. In certain embodiments, SSPE (1×SSPE is 0.15 M NaCl, 10 mM NaH2PO4, and 1.25 mM EDTA, pH 7.4) can be substituted for SSC (1×SSPE is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm (° C.)=2(# of A+T bases)+4(# of G+C bases). For hybrids between 18 and 49 base pairs in length, Tm (° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to the hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS) chelating agents (e.g., EDTA), Ficoll, PVP and the like. When using nylon membranes, in particular, an additional, non-limiting example of stringent hybridization conditions is hybridization in 0.25-0.5M NaH2PO4, 7% SDS at about 65° C., followed by one or more washes at 0.02M NaH2PO4, 1% SDS at 65° C. or alternatively 0.2×SSC, 1% SDS (see, e.g., Church and Gilbert, 1984).

Thus, as generally set forth above, certain embodiments of the disclosure are related to variant strains of filamentous fungus cells comprise a genetic modification of a gene encoding a native GEF1 protein. As used herein, the terms “modification” and “genetic modification” are used interchangeably and include, but are not limited to: (a) the introduction, substitution, or removal of one or more nucleotides in a gene, or the introduction, substitution, or removal of one or more nucleotides in a regulatory element required for the transcription or translation of the gene, (b) gene disruption, (c) gene conversion, (d) gene deletion, (e) the down-regulation of a gene (e.g., antisense RNA, siRNA, miRNA, and the like), (f) specific mutagenesis (including, but not limited to, CRISPR/Cas9 based mutagenesis) and/or (g) random mutagenesis of any one or more the genes disclosed herein.

As used herein, a variant strain of filamentous fungus comprising a genetic modification includes, but is not limited to a genetic modification of a gene encoding a native GEF1 protein disclosed herein.

Thus, as described in further detail below, various molecular biological methods are well known and available to one skilled in the art for generating/constructing such variant strains of filamentous fungus cells.

As used herein, “the introduction, substitution, or removal of one or more nucleotides in a gene encoding a protein”, such genetic modifications include the gene's coding sequence (i.e., exons) and non-coding intervening (introns) sequences.

As used herein, “disruption of a gene”, “gene disruption”, “inactivation of a gene” and “gene inactivation” are used interchangeably and refer broadly to any genetic modification that substantially disrupts/inactivates a target gene. Exemplary methods of gene disruptions include, but are not limited to, the complete or partial deletion of any portion of a gene, including a polypeptide coding sequence (CDS), a promoter, an enhancer, or another regulatory element, or mutagenesis of the same, where mutagenesis encompasses substitutions, insertions, deletions, inversions, and any combinations and variations thereof which disrupt/inactivate the target gene(s) and substantially reduce or prevent the expression/production of the functional gene product. In certain embodiments of the disclosure, such gene disruptions prevent a host cell from expressing/producing the encoded lov gene product.

In certain embodiments, a gene, polynucleotide or nucleic acid sequence encoding a native GEF1 protein is genetically modified using an established gene editing technique, such as CRISPR/Cas9 gene editing, zinc-finger nuclease (ZFN) gene editing, transcription activator-like effector nuclease editing (TALEN), homing (mega) nuclease editing, and the like.

In other embodiments, a variant strain of filamentous fungus is constructed (i.e., genetically modified) by the process of gene conversion (e.g., see Iglesias and Trautner, 1983).

In other embodiments, a protein of interest (e.g., an endogenous POI or a heterologous POI) expressed/produced by the fungal cells of the disclosure is detected, measured, assayed and the like, by protein quantification methods, gene transcription methods, mRNA translation methods and the like, including, but not limited to protein migration/mobility (SDS-PAGE), mass spectrometry, HPLC, size exclusion, ultracentrifugation sedimentation velocity analysis, transcriptomics, proteomics, fluorescent tags, epitope tags, fluorescent protein (GFP, RFP, etc.) chimeras/hybrids and the like.

As used herein, functionally and/or structurally similar proteins are considered to be “related proteins”. Such related proteins can be derived from organisms of different genera and/or species, or even different classes of organisms (e.g., bacteria and fungi). Related proteins also encompass homologues and/or orthologues determined by primary sequence analysis, determined by secondary or tertiary structure analysis, or determined by immunological cross-reactivity.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or even comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “operably linked” as used herein refers to the association of nucleic acid sequences on a single nucleic acid fragment so that the function of one is affected by the other. For example, a promoter is operably linked with a coding sequence (e.g., an ORF) when it is capable of affecting the expression of that coding sequence (i.e., that the coding sequence is under the transcriptional control of the promoter).

Coding sequences can be operably linked to regulatory sequences in sense or antisense orientation. A nucleic acid is “operably linked” when it is placed into a functional relationship with another nucleic acid sequence. For example, DNA encoding a secretory leader (i.e., a signal peptide), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As defined herein, the term “introducing”, as used in phrases such as “introducing into a fungal cell” at least one polynucleotide open reading frame (ORF), or a gene thereof, or a vector thereof, includes methods known in the art for introducing polynucleotides into a cell, including, but not limited to protoplast fusion, natural or artificial transformation (e.g., calcium chloride, electroporation), transduction, transfection and the like.

As used herein, “transformed” or “transformation” mean a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in the cell that is to be transformed).

As used herein, “transformation” refers to introducing an exogenous DNA into a host cell so that the DNA is maintained as a chromosomal integrant or a self-replicating extra-chromosomal vector. As used herein, “transforming DNA”, “transforming sequence”, and “DNA construct” refer to DNA that is used to introduce sequences into a host cell. The DNA may be generated in vitro by PCR or any other suitable techniques. In some embodiments, the transforming DNA comprises an incoming sequence, while in other embodiments it further comprises an incoming sequence flanked by homology boxes. In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein “an incoming sequence” refers to a DNA sequence that is introduced into the fungal cell chromosome. In some embodiments, the incoming sequence is part of a DNA construct. In other embodiments, the incoming sequence encodes one or more proteins of interest. In some embodiments, the incoming sequence comprises a sequence that may or may not already be present in the genome of the cell to be transformed (i.e., it may be either a homologous or heterologous sequence). In some embodiments, the incoming sequence encodes one or more proteins of interest, a gene, and/or a mutated or modified gene.

In alternative embodiments, the incoming sequence encodes a functional wild-type gene or operon, a functional mutant gene or operon, or a nonfunctional gene or operon. In some embodiments, an incoming sequence is a non-functional sequence inserted into a gene to disrupt function of the gene. In another embodiment, the incoming sequence includes a selective marker. In a further embodiment the incoming sequence includes two homology boxes.

As used herein, “homology box” refers to a nucleic acid sequence, which is homologous to a sequence in the fungal cell chromosome. More specifically, a homology box is an upstream or downstream region having between about 80 and 100% sequence identity, between about 90 and 100% sequence identity, or between about 95 and 100% sequence identity with the immediate flanking coding region of a gene or part of a gene to be deleted, disrupted, inactivated, down-regulated and the like, according to the invention. These sequences direct where in the fungal cell chromosome a DNA construct is integrated and directs what part of the fungal cell chromosome is replaced by the incoming sequence. While not meant to limit the present disclosure, a homology box may include about between 1 base pair (bp) to 200 kilobases (kb). Preferably, a homology box includes about between 1 bp and 10.0 kb; between 1 bp and 5.0 kb; between 1 bp and 2.5 kb; between 1 bp and 1.0 kb, and between 0.25 kb and 2.5 kb. A homology box may also include about 10.0 kb, 5.0 kb, 2.5 kb, 2.0 kb, 1.5 kb, 1.0 kb, 0.5 kb, 0.25 kb and 0.1 kb. In some embodiments, the 5′ and 3′ ends of a selective marker are flanked by a homology box wherein the homology box comprises nucleic acid sequences immediately flanking the coding region of the gene.

As used herein, the term “selectable marker-encoding nucleotide sequence” refers to a nucleotide sequence which is capable of expression in the host cells and where expression of the selectable marker confers to cells containing the expressed gene the ability to grow in the presence of a corresponding selective agent or lack of an essential nutrient.

As used herein, the terms “selectable marker” and “selective marker” refer to a nucleic acid (e.g., a gene) capable of expression in host cell which allows for ease of selection of those hosts containing the vector. Examples of such selectable markers include, but are not limited to, antimicrobials. Thus, the term “selectable marker” refers to genes that provide an indication that a host cell has taken up an incoming DNA of interest or some other reaction has occurred. Typically, selectable markers are genes that confer antimicrobial resistance or a metabolic advantage on the host cell to allow cells containing the exogenous DNA to be distinguished from cells that have not received any exogenous sequence during the transformation.

As defined herein, a host cell “genome”, a fungal cell “genome”, or a filamentous fungus cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “vector” refers to any nucleic acid that can be replicated (propagated) in cells and can carry new genes or DNA segments (e.g., an “incoming sequence”) into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously) or can integrate into the chromosome of a host cell.

A used herein, a “transformation cassette” refers to a specific vector comprising a gene (or ORF thereof), and having elements in addition to the gene that facilitate transformation of a particular host cell.

As used herein, “expression vector” refers to a vector that has the ability to incorporate and express heterologous DNA in a cell. Many prokaryotic and eukaryotic expression vectors are commercially available and know to one skilled in the art. Selection of appropriate expression vectors is within the knowledge of one skilled in the art.

As used herein, the terms “expression cassette” and “expression vector” refer to a nucleic acid construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell. In certain embodiments, a DNA construct of the disclosure comprises a selective marker and an inactivating chromosomal or gene or DNA segment as defined herein.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing genetic modifications into the chromosome of a host cell through homologous recombination.

In some embodiments, a targeting vector comprises other non-homologous sequences, e.g., added to the ends (i.e., stuffer sequences or flanking sequences). The ends can be closed such that the targeting vector forms a closed circle, such as, for example, insertion into a vector.

As used herein, a variant cell (or strain) comprising an “enhanced protein productivity phenotype” includes, but is not limited to, a variant cell (or strain) comprising an enhanced/increased volumetric productivity, a variant cell (or strain) comprising an enhanced/increased carbon conversion efficiency, a variant cell (or strain) comprising an enhanced/increased protein yield, a variant cell (or strain) comprising an enhanced/increased specific protein productivity and the like. For example, in certain embodiments, a variant cell or strain comprising an enhanced protein productivity phenotype expresses/produces at least 0.1% or more total protein (g) per g of fed sugars (relative to parental strain), wherein fed sugars can be expressed in terms of mass of sugar added to the fermentor during production phase (i.e., following feed-start).

As defined herein, the phrases “enhanced protein productivity phenotype” and “increased protein productivity phenotype”, may be used interchangeably.

As used herein, when describing an “enhanced/increased protein productivity phenotype” in an unmodified (parental) cell vis-à-vis the modified (variant/daughter), it will be understood that the “parental” and “variant” cells are grown/cultivated/fermented under the same conditions (e.g., the same conditions such as media, temperature, pH and the like). Similarly, when describing the “expression/production” of a protein of interest (POI) in an unmodified (parental) cell vis-à-vis the “expression/production” of the same POI in a modified (variant/daughter) cell, it will be understood that the “parental” and “variant” cells are grown/cultivated/fermented under essentially the same conditions (e.g., the same conditions such as media, temperature, pH and the like).

As used herein, “aerobic fermentation” refers to growth in the presence of oxygen.

As used herein, the terms “broth”, “cell broth”, “fermentation broth” and/or “culture broth” are used interchangeably, and refer collectively to (i) the fermentation (culture) medium and (ii) the cells, in a liquid (submerged) culture.

As used herein, the term “cell mass” refers to the cell component (including intact and lysed cells) present in a liquid (submerged) culture. Cell mass can be expressed in dry cell weight (DCW) or wet cell weight (WCW).

### II. Fungal Strains Comprising Enhanced Protein Productivity Phenotypes at Elevated Cultivation Temperatures

As generally described and set forth below in the Examples section, Applicant serially propagated a Trichoderma reesei whole cellulase strain named T4 under selective conditions to identify and isolate mutant strains thereof capable of high temperature protein production (i.e., without adversely affecting specific productivity (Qₚ); e.g., see Example 1). More particularly, a mutant T. reesei strain named “T4-E1” was identified and isolated under such selective conditions (Example 2), wherein the mutant T4-E1 strain has a similar specific productivity at 28° C., relative to the parental T4 strain at 25° C. (TABLE 1). Applicant identified the mutated gene in the T4-E1 strain, wherein the deduced amino acid sequence of the encoded protein (SEQ ID NO: 2; PID:120482) includes a region of sequence homology with a GEF1 protein domain (SEQ ID NO: 5).

As detailed in Example 2, the mutation in the T4-E1 strain replaced a glutamate codon (Glu; amino acid position 1,245) with a pre-mature stop codon, which thereby truncated the (GEF1) protein near the beginning of the “GEF1 domain” (e.g., see FIG. 3A native sequence versus FIG. 3B C-terminally truncated sequence).

As described in Example 3 of the disclosure, Applicant further constructed a variant (modified) Trichoderma strain (named “T4-ΔRho #22D”) derived from the T4 parental strain, wherein the variant T4-ΔRho #22D strain comprises a pyr2 marker gene introduced at the Cas9 cleavage site (TS3), thereby disrupting the GEF1 coding sequence. For example, the fermenter (cultivation) performance of the wild-type (parental) T4 strain, the mutant T4-E1 strain and the constructed variant T4-ΔRho #22D strain are presented in TABLE 1, wherein the mutant T4-E1 strain and the constructed variant T4-ΔRho #22D strain demonstrate increased total protein production rates at 28° C., relative to the parental T4 strain total protein production rate at 25° C.

As further described in Example 4, Applicant constructed a Trichoderma sp. production strain transformed with copies of the gene encoding a S. coccosporum DXST (STCE1) endoglucanase. More particularly, a parental Trichoderma DXST production strain named “t-ATV84” was transformed with copies of the gene encoding a S. coccosporum DXST endoglucanase and further comprises deletions of native cellulase genes encoding the CBHI, CBHII, EGI and EGII proteins. Likewise, the variant (modified) Trichoderma DXST production strain named “t-AWC88” was derived from the t-ATV84 strain, and further comprises an introduced disruption of the GEF1 gene. For example, as shown in TABLE 2, the variant Trichoderma strain (t-AWC88) comprising the disrupted GEF1 gene demonstrates a higher amount of DXST endoglucanase produced at 25° C., 26° C. and 27° C., relative to the amount of DXST endoglucanase produced by the parental strain (t-ATV84) at 25° C., which comprises the native GEF1 gene.

### III. Molecular Biology

As generally set forth above, certain embodiments of the disclosure are related to modified filamentous fungal strains comprising enhanced protein productivity phenotypes. In certain preferred embodiments, the modified (variant) filamentous fungal strains comprise enhanced protein productivity phenotypes at elevated fermentation (cultivation) temperatures. In another embodiment, the disclosure is related to modified yeast strains comprising enhanced ethanol productivity phenotypes. In certain preferred embodiments, the modified (variant) yeast strains comprise enhanced ethanol productivity phenotypes at elevated fermentation (cultivation) temperatures.

Thus, certain other embodiments of the disclosure are related to molecular biology, genetic modifications, polynucleotides, genes, ORFs, vectors, expression cassettes, and the like. In certain embodiments, the disclosure is related to recombinant nucleic acids (polynucleotides) comprising a gene or ORF encoding a protein of interest (POI). In certain embodiments, a recombinant nucleic acid (polynucleotide) comprises a polynucleotide expression cassette encoding a POI.

In certain embodiments, a polynucleotide of the disclosure comprises one or more selectable markers. Selectable markers for use in filamentous fungi include, but are not limited to, als1, amdS, hygR, pyr2, pyr4, pyrG, sucA, a bleomycin resistance marker, a blasticidin resistance marker, a pyrithiamine resistance marker, a chlorimuron ethyl resistance marker, a neomycin resistance marker, an adenine pathway gene, a tryptophan pathway gene, a thymidine kinase marker and the like. In a particular embodiment, the selectable marker is pyr2, which compositions and methods of use are generally set forth in PCT Publication No. WO2011/153449.

Standard techniques for transformation of filamentous fungi and culturing the fungi (which are well known to one skilled in the art) are used to transform a fungal host cell of the disclosure. Thus, the introduction of a DNA construct or vector into a fungal host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated micro-projectiles, gene gun or biolistic transformation, protoplast fusion and the like. General transformation techniques are known in the art (see, e.g., Ausubel et al., 1987, Sambrook et al., 2001 and 2012, and Campbell et al., 1989). The expression of heterologous proteins in Trichoderma is described, for example, in U.S. Pat. Nos. 6,022,725; 6,268,328; Harkki et al., 1991 and Harkki et al., 1989. Reference is also made to Cao et al. (2000) for transformation of Aspergillus strains.

Generally, transformation of Trichoderma sp. uses protoplasts or cells that have been subjected to a permeability treatment, typically at a density of 10⁵ to 10⁷/mL, particularly 2×10⁶/mL. A volume of 100 L of these protoplasts or cells in an appropriate solution (e.g., 1.2 M sorbitol and 50 mM CaCl₂) is mixed with the desired DNA. Generally, a high concentration of polyethylene glycol (PEG) is added to the uptake solution. Additives, such as dimethyl sulfoxide, heparin, spermidine, potassium chloride and the like, may also be added to the uptake solution to facilitate transformation. Similar procedures are available for other fungal host cells (e.g., see U.S. Pat. Nos. 6,022,725 and 6,268,328, both of which are incorporated by reference.

Thus, the methods and compositions of instant disclosure generally rely on routine techniques in the field of recombinant genetics. For example, in certain embodiments, a heterologous gene or ORF encoding a protein of interest is introduced into a filamentous fungal (host) cell. In certain embodiments, the heterologous gene or ORF is typically cloned into an intermediate vector, before being transformed into a filamentous fungal (host) cells for replication and/or expression. These intermediate vectors can be prokaryotic vectors, such as, e.g., plasmids, or shuttle vectors. In certain embodiments, the expression of the heterologous gene or ORF is under the control of its native promoter. In other embodiments, the expression of the heterologous gene or ORF is placed under the control of a heterologous promoter, which can be a heterologous constitutive promoter or a heterologous inducible promoter.

Those skilled in the art are aware that a natural (native) promoter can be modified by replacement, substitution, addition or elimination of one or more nucleotides, without changing its function. The practice of the invention encompasses but is not constrained by such alterations to the promoter.

The expression vector typically contains a transcription unit or “expression cassette” that contains all the additional elements required for the expression of the heterologous sequence. For example, a typical expression cassette contains a 5′ promoter operably linked to a heterologous nucleic acid sequence encoding a protein of interest and may further comprise sequence signals required for efficient polyadenylation of the transcript, ribosome binding sites, and translation termination sequences. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes. Although any fungal terminator is likely to be functional in the present invention, preferred terminators include: the terminator from Trichoderma cbhI gene, the terminator from Aspergillus nidulans trpC gene (Yelton et al., 1984; Mullaney et al., 1985) and the Aspergillus awamori or Aspergillus niger glucoamylase genes (Nunberg et al., 1984; Boel et al., 1984).

The particular expression vector used to transport the genetic information into the cell is not particularly critical. Any of the conventional vectors used for expression in eukaryotic or prokaryotic cells may be used. Standard bacterial expression vectors include bacteriophages λ and M13, as well as plasmids such as pBR322 based plasmids, pSKF, pET23D, and fusion expression systems such as MBP, GST, and LacZ. Epitope tags can also be added to recombinant proteins to provide convenient methods of isolation, e.g., c-myc.

The elements that can be included in expression vectors may also be a replicon, a gene encoding antibiotic resistance to permit selection of bacteria that harbor recombinant plasmids, or unique restriction sites in nonessential regions of the plasmid to allow insertion of heterologous sequences. The particular antibiotic resistance gene chosen is not dispositive either, as any of the many resistance genes known in the art may be suitable. The prokaryotic sequences are preferably chosen such that they do not interfere with the replication or integration of the DNA in Trichoderma reesei.

The methods of transformation of the present invention may result in the stable integration of all or part of the transformation vector into the genome of the filamentous fungus. However, transformation resulting in the maintenance of a self-replicating extra-chromosomal transformation vector is also contemplated. Many standard transfection methods can be used to produce Trichoderma reesei cell lines that express large quantities of the heterologous protein, and as such, any of the known procedures for introducing foreign nucleotide sequences into fungal host cells may be used. These include the use of calcium phosphate transfection, polybrene, protoplast fusion, electroporation, biolistics, liposomes, microinjection, plasma vectors, viral vectors and any of the other known methods for introducing cloned genomic DNA, cDNA, synthetic DNA or other foreign genetic material into a host cell. Also of use is the Agrobacterium-mediated transfection method such as the one described in U.S. Pat. No. 6,255,115.

After the expression vector is introduced into the cells, the transformed cells are cultured under conditions favoring expression of gene. Large batches of transformed cells can be cultured as described herein. Finally, the protein product is recovered from the culture using standard techniques. Thus, the disclosure herein provides for the expression and enhanced production of desired proteins of interest, particularly at elevated fermentation (cultivation) temperatures described herein.

In certain other embodiments, the disclosure is related genetically modified filamentous fungal strains (cells) comprising enhanced protein productivity phenotypes. In particular embodiments, a modified fungal strain of the disclosure comprised an enhanced protein productivity phenotype at elevated fermentation temperatures. For example, in certain embodiments, a variant strain of filamentous fungus comprises genetic modification of a gene encoding a GEF1 protein, wherein the genetic modification includes, but is not limited to: (a) the introduction, substitution, or removal of one or more nucleotides in the GEF1 gene (or ORF thereof), or the introduction, substitution, or removal of one or more nucleotides in a regulatory element required for the transcription or translation of the GEF1 gene (or ORF thereof), (b) a gene disruption, (c) a gene conversion, (d) a gene deletion, (e) a gene down-regulation, (f) specific mutagenesis and/or (g) random mutagenesis of a gene encoding a GEF1 protein comprising sequence identity to SEQ ID NO: 2.

Thus, in certain embodiments, a variant strain of filamentous fungus comprising a genetic modification is constructed by gene deletion to eliminate the expression/production of the GEF1 protein.

In other embodiments, a variant strain of filamentous fungus comprising a genetic modification is constructed by partial gene deletion or gene disruption to eliminate the expression/production of the native GEF1 protein. For example, as set forth below in the Example 3 (FIG. 3B), inactivation of the native GEF1 gene in a parental filamentous fungal strain, by introducing a pre-mature stop codon (i.e., which C-terminally truncates the GEF1 protein; SEQ ID NO: 4), resulted in a variant (daughter) strain comprising an enhanced protein productivity phenotype relative to the parental cell when fermented at 25° C. More particularly, when the parental (T4) and daughter (T4-ΔRho #22D) strains were fermented at an elevated fermentation temperature (28° C.), the variant (daughter) strain demonstrated an increased protein productivity phenotype (TABLE 1, protein production rate) relative to the parental strain.

Thus, in certain embodiments, a modified filamentous fungal strain comprises a partial deletion of the GEF1 gene, wherein a partial deletion includes the partial deletion of any portion of the GEF1 gene's coding sequence, wherein such variant strain comprises an enhanced protein productivity phenotype. Thus, in certain other embodiments, such variant strains do not express/produce the GEF1 protein, or such variant strains express/produce a reduced amount of the GEF1 protein relative to the parental strain.

Thus, as generally set forth herein and described above, one skilled in the art may readily perform one or more genetic modifications and construct variant filamentous fungus strains thereof, by reference to one or more nucleic acid sequences and/or protein sequence disclosed herein.

For example, gene deletion techniques enable the partial or complete removal of the gene, thereby eliminating or reducing expression/production of the protein, and/or thereby eliminating or reducing expression/production the encoded protein (e.g., GEF1). In such methods, the deletion of the gene may be accomplished by homologous recombination using an integration plasmid/vector that has been constructed to contiguously contain the 5′ and 3′ regions flanking the gene. The contiguous 5′ and 3′ regions may be introduced into a filamentous fungal cell, for example, on an integrative plasmid/vector in association with a selectable marker to allow the plasmid to become integrated in the cell.

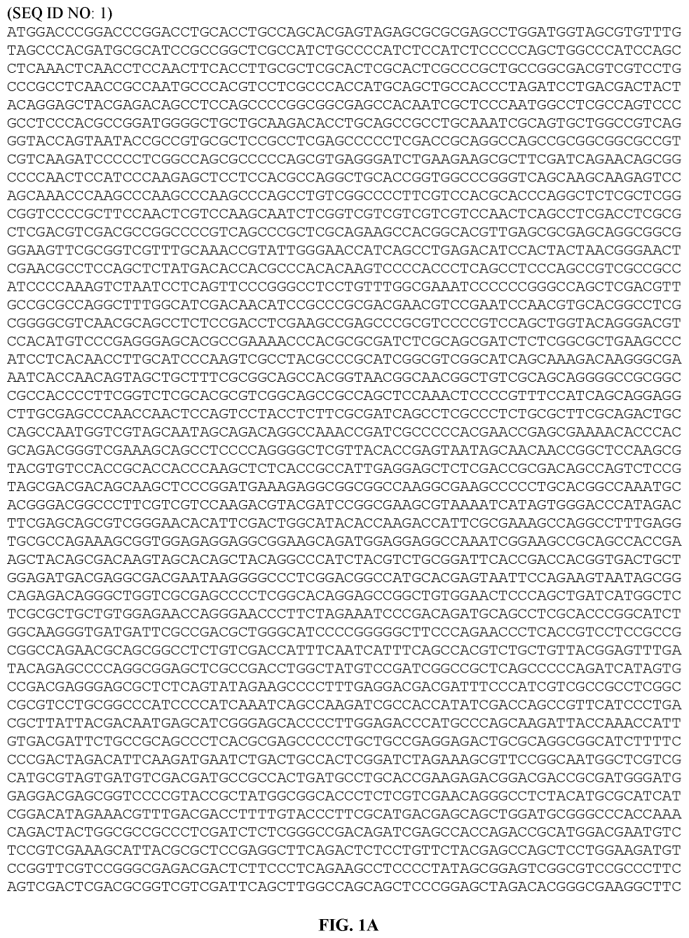

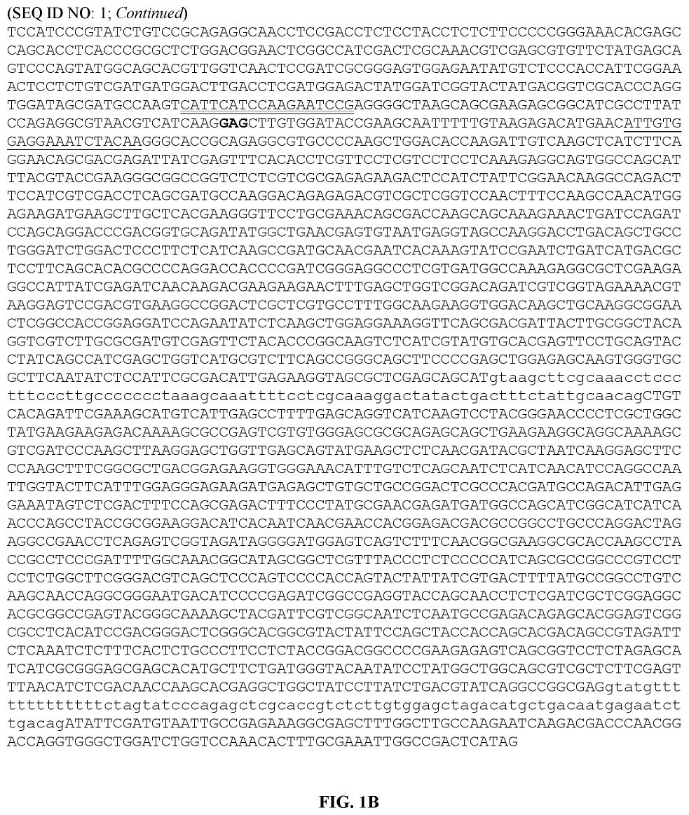

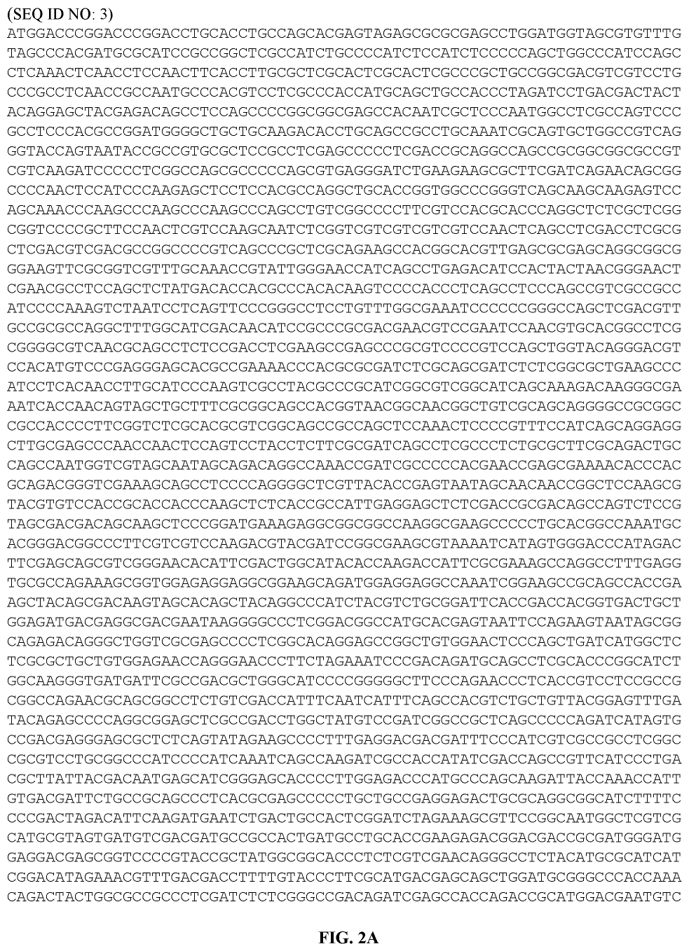

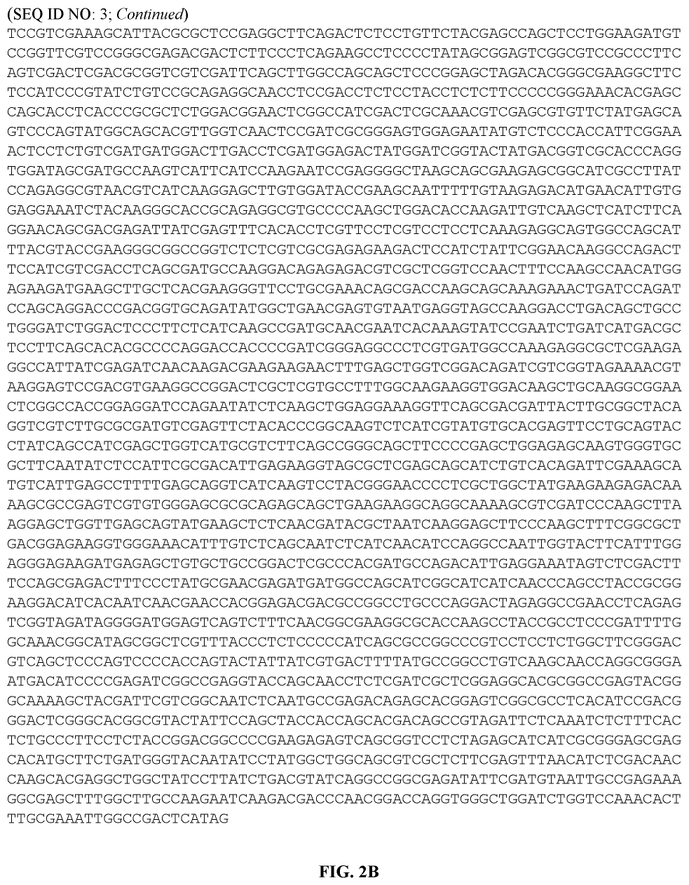

In other embodiments, a variant strain of filamentous fungus comprises genetic modification which disrupts or inactivates the gene encoding the protein (e.g., GEF1). Exemplary methods of gene disruption/inactivation include disrupting any portion of the gene (e.g., see, FIG. 1-FIG. 3), including the polypeptide coding sequence (CDS), promoter, enhancer, or another regulatory element, which disruption includes substitutions, insertions, deletions, inversions, and combinations thereof and variations thereof. A non-limiting example of a gene disruption technique includes inserting (integrating) into one or more of the genes of the disclosure an integrative plasmid containing a nucleic acid fragment homologous to the (e.g., GEF1) gene, which will create a duplication of the region of homology and incorporate (insert) vector DNA between the duplicated regions. In certain other non-limiting examples, a gene disruption technique includes inserting into a gene (e.g., a gene encoding a GEF1 protein) an integrative plasmid containing a nucleic acid fragment homologous to the (e.g., GEF1) gene, which will create a duplication of the region of homology and incorporate (insert) vector DNA between the duplicated regions, wherein the vector DNA inserted separates, e.g., the promoter of the GEF1 gene from the GEF1 protein coding region, or interrupts (disrupts) the coding, or non-coding, sequence of the GEF1 gene, resulting in an enhanced protein productivity phenotype. Thus, a disrupting construct may be a selectable marker gene (e.g., pyr2) accompanied by 5′ and 3′ regions homologous to the GEF1 gene. The selectable marker enables identification of transformants containing the disrupted gene. Thus, in certain embodiments, gene disruption includes modification of control elements of the gene, such as the promoter, ribosomal binding site (RBS), untranslated regions (UTRs), codon changes, and the like.

In other embodiments, a variant strain of filamentous fungus is constructed (i.e., genetically modified) by introducing, substituting, or removing one or more nucleotides in the gene, or a regulatory element required for the transcription or translation thereof. For example, nucleotides may be inserted or removed so as to result in the introduction of a pre-mature stop codon, the removal of the start codon, or a frame-shift of the open reading frame (ORF). Such a modification may be accomplished by site-directed mutagenesis or PCR generated mutagenesis in accordance with methods known in the art (e.g., see, Botstein and Shortle, 1985; Lo et al., 1985; Higuchi et al., 1988; Shimada, 1996; Ho et al., 1989; Horton et al., 1989 and Sarkar and Sommer, 1990).

In another embodiment, a variant strain of filamentous fungus is constructed by the process of gene conversion (e.g., see Iglesias and Trautner, 1983). For example, in the gene conversion method, a nucleic acid sequence corresponding to the target gene is mutagenized in vitro to produce a defective nucleic acid sequence, which is then transformed into the parental cell to produce a variant cell comprising a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (Perego, 1993).

In other embodiments, a variant strain of filamentous fungus is constructed by established anti-sense (gene-silencing) techniques, using a nucleotide sequence complementary to the nucleic acid sequence of the GEF1 gene (Parish and Stoker, 1997). More specifically, expression of a gene by a filamentous fungus strain may be reduced (down-regulated) or eliminated by introducing a nucleotide sequence complementary to the nucleic acid sequence of the gene, which is transcribed in the cell and is capable of hybridizing to the mRNA produced in the cell. Under conditions allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, and the like, all of which are well known to the skilled artisan.

In other embodiments, a variant strain of filamentous fungus is constructed by random or specific mutagenesis using methods well known in the art, including, but not limited to, chemical mutagenesis and transposition (see, e.g., Youngman et al., 1983). Modification of the gene may be performed by subjecting the parental cell to mutagenesis and screening for mutant cells in which expression of the GEF1 gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods. Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

In certain other embodiments, a variant strain of filamentous fungus is constructed by means of site specific gene editing techniques. For example, in certain embodiments, a variant strain of filamentous fungus is constructed (i.e., genetically modified) by use of transcriptional activator like endonucleases (TALENs), zinc-finger endonucleases (ZFNs), homing (mega) endonuclease and the like. More particularly, the portion of the gene to be modified (e.g., a coding region, a non-coding region, a leader sequence, a pro-peptide sequence, a signal sequence, a transcription terminator, a transcriptional activator, or other regulatory elements required for expression of the coding region) is subjected genetic modification by means of ZFN gene editing, TALEN gene editing, homing (mega) endonuclease and the like, which modification methods are well known and available to one skilled in the art.

In certain other embodiments, a variant strain of filamentous fungus is constructed by means of CRISPR/Cas9 editing (e.g., see Examples herewith). More specifically, compositions and methods for fungal genome modification by CRISPR/Cas9 systems are described and well known in the art (e.g., see, PCT Publication Nos: WO2016/100571, WO2016/100568, WO2016/100272, WO2016/100562 and the like). Thus, a gene encoding a GEF1 protein can be disrupted, deleted, mutated or otherwise genetically modified by means of nucleic acid guided endonucleases, that find their target DNA by binding either a guide RNA (e.g., Cas9) or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (e.g., a Cas9 from S. pyogenes, or a codon optimized gene encoding the Cas9 nuclease) is operably linked to a promoter active in the filamentous fungal cell and a terminator active in filamentous fungal cell, thereby creating a filamentous fungal Cas9 expression cassette. Likewise, one or more target sites unique to the gene of interest are readily identified by a person skilled in the art.

For example, to build a DNA construct encoding a gRNA-directed to a target site within the gene of interest, the variable targeting domain (VT) will comprise nucleotides of the target site which are 5′ of the (PAM) proto-spacer adjacent motif (TGG), which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a filamentous fungal expression cassette for the gRNA is created by operably linking the DNA encoding the gRNA to a promoter active in filamentous fungal cells and a terminator active in filamentous fungal cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the filamentous fungal host's machinery to repair the DNA break generated by the RGEN (RNA-guided endonuclease).

The Cas9 expression cassette, the gRNA expression cassette and the editing template can be co-delivered to filamentous fungal cells using many different methods (e.g., protoplast fusion, electroporation, natural competence, or induced competence). The transformed cells are screened by PCR, by amplifying the target locus with a forward and reverse primer. These primers can amplify the wild-type locus or the modified locus that has been edited by the RGEN. These fragments are then sequenced using a sequencing primer to identify edited colonies.

Another way in which a gene encoding a GEF1 protein of the disclosure can be genetically modified is by altering the expression level of the gene of interest. For example, nuclease-defective variants of such nucleotide-guided endonucleases (e.g., Cas9 D10A, N863A or Cas9 D10A, H840A) can be used to modulate gene expression levels by enhancing or antagonizing transcription of the target gene. These Cas9 variants are inactive for all nuclease domains present in the protein sequence, but retain the RNA-guided DNA binding activity (i.e., these Cas9 variants are unable to cleave either strand of DNA when bound to the cognate target site). Thus, the nuclease-defective proteins (i.e., Cas9 variants) can be expressed as a filamentous fungus expression cassette and when combined with a filamentous fungus gRNA expression cassette, such that the Cas9 variant protein is directed to a specific target sequence within the cell. The binding of the Cas9 (variant) protein to specific gene target sites can block the binding or movement of transcription machinery on the DNA of the cell, thereby decreasing the amount of a gene product produced. Thus, any of the genes disclosed herein can be targeted for reduced gene expression using this method. Gene silencing can be monitored in cells containing the nuclease-defective Cas9 expression cassette and the gRNA expression cassette(s) by using methods such as RNAseq.

### V. Proteins of Interest

As briefly stated in the preceding sections, the present strains and methods find use in the production of commercially important proteins in submerged cultures of filamentous fungi. A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

In certain embodiments, a variant strain of filamentous fungus exhibits an increased protein titer relative to the (unmodified) parental strain, wherein protein titer is defined as the amount of protein per volume (g/L). For example, titers can be measured by methods known in the art (e.g., ELISA, HPLC, Bradford assay, LC/MS and the like). Thus, in certain embodiments, a variant strain of filamentous fungus comprises a protein titer increase of at least about 0.1%, at least about 1%, at least about 5%, at least about 6%, at least about 7%, at least about 8%, at least about 9%, or at least about 10% or more as compared to the unmodified (parental) cell.

In certain embodiments, a variant strain of filamentous fungus exhibits an increased volumetric productivity relative to the (unmodified) parental strain, wherein volumetric productivity is defined as the amount of protein produced (g) during the fermentation per nominal volume (L) of the bioreactor per total fermentation time (h). For example, volumetric productivities can be measured by methods know in the art (e.g., ELISA, HPLC, Bradford assay, LC/MS and the like). Thus, in certain embodiments, a variant strain of filamentous fungus comprises a volumetric productivity increase of at least about 0.1%, at least about 1%, at least about 5%, at least about 6%, at least about 7%, at least about 8%, at least about 9%, or at least about 10% or more as compared to the unmodified (parental) cell.

In certain other embodiments, a variant strain of filamentous fungus exhibits an increased total protein yield, wherein total protein yield is defined as the amount of protein produced (g) per gram of carbohydrate fed, relative to the (unmodified) parental strain. Thus, as used herein, total protein yield (g/g) may be calculated using the following equation:

Y_{f}=Tₚ/T_{c}

wherein “Y_{f}” is total protein yield (g/g), “Tₚ” is the total protein produced during the fermentation (g) and “T_{c}” is the total carbohydrate (g) fed during the fermentation (bioreactor) run. In certain embodiments, the increase in total protein yield of the modified strain (i.e., relative to the parental strain) is an increase of at least about 0.1%, at least about 1%, at least about 5%, at least about 6%, at least about 7%, at least about 8%, at least about 9%, or at least about 10% or more as compared to the unmodified (parental) cell.

Total protein yield may also be described as carbon conversion efficiency/carbon yield, for example, as in the percentage (%) of carbon fed that is incorporated into total protein. Thus, in certain embodiments, a variant strain of filamentous fungus comprises an increased carbon conversion efficiency (e.g., an increase in the percentage (%) of carbon fed that is incorporated into total protein), relative to the (unmodified) parental strain. In certain embodiments, the increase in carbon conversion efficiency of the modified strain (i.e., relative to the parental strain) is an increase of at least about 0.1%, at least about 1%, at least about 5%, at least about 6%, at least about 7%, at least about 8%, at least about 9%, or at least about 10% or more as compared to the unmodified (parental) cell.

In certain embodiments, a variant strain of filamentous fungus exhibits an increased specific productivity (Qp) of a POI relative the (unmodified) parental strain. For example, the detection of specific productivity (Qp) is a suitable method for evaluating rate of protein production. The specific productivity (Qₚ) can be determined using the following equation:

“Qp=gP/gDCW·hr”

wherein, “gP” is grams of protein produced in the tank; “gDCW” is grams of dry cell weight (DCW) in the tank and “hr” is fermentation time in hours from the time of inoculation, which includes the time of production as well as growth time. Thus, in certain embodiments, a variant strain of filamentous fungus comprises a specific productivity (Qp) increase of at least about 0.1%, at least about 1%, at least about 5%, at least about 6%, at least about 7%, at least about 8%, at least about 9%, or at least about 10% or more as compared to the unmodified (parental) cell.

In certain embodiments, a POI or a variant POI thereof is selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lyases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannanases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

In certain embodiments, a POI or a variant POI thereof is selected from an Enzyme Commission (EC) Number selected from the group consisting of EC 1, EC 2, EC 3, EC 4, EC 5 or EC 6.

For example, in certain embodiments a POI is an oxidoreductase enzyme, including, but not limited to, an EC1 (oxidoreductase) enzyme selected from EC 1.10.3.2 (e.g., a laccase), EC 1.10.3.3 (e.g., L-ascorbate oxidase), EC 1.1.1.1 (e.g., alcohol dehydrogenase), EC 1.11.1.10 (e.g., chloride peroxidase), EC 1.11.1.17 (e.g., peroxidase), EC 1.1.1.27 (e.g., L-lactate dehydrogenase), EC 1.1.1.47 (e.g., glucose 1-dehydrogenase), EC 1.1.3.X (e.g., glucose oxidase), EC 1.1.3.10 (e.g., pyranose oxidase), EC 1.13.11.X (e.g., dioxygenase), EC 1.13.11.12 (e.g., lineolate 13S-lipozygenase), EC 1.1.3.13 (e.g., alcohol oxidase), EC 1.14.14.1 (e.g., monooxygenase), EC 1.14.18.1 (e.g., monophenol monooxigenase), EC 1.15.1.1 (e.g., superoxide dismutase), EC 1.1.5.9 (formerly EC 1.1.99.10, e.g., glucose dehydrogenase), EC 1.1.99.18 (e.g., cellobiose dehydrogenase), EC 1.1.99.29 (e.g., pyranose dehydrogenase), EC 1.2.1.X (e.g., fatty acid reductase), EC 1.2.1.10 (e.g., acetaldehyde dehydrogenase), EC 1.5.3.X (e.g., fructosyl amine reductase), EC 1.8.1.X (e.g., disulfide reductase) and EC 1.8.3.2 (e.g., thiol oxidase).

In certain embodiments a POI is a transferase enzyme, including, but not limited to, an EC 2 (transferase) enzyme selected from EC 2.3.2.13 (e.g., transglutaminase), EC 2.4.1.X (e.g., hexosyltransferase), EC 2.4.1.40 (e.g., alternasucrase), EC 2.4.1.18 (e.g., 1,4 alpha-glucan branching enzyme), EC 2.4.1.19 (e.g., cyclomaltodextrin glucanotransferase), EC 2.4.1.2 (e.g., dextrin dextranase), EC 2.4.1.20 (e.g., cellobiose phosphorylase), EC 2.4.1.25 (e.g., 4-alpha-glucanotransferase), EC 2.4.1.333 (e.g., 1,2-beta-oligoglucan phosphor transferase), EC 2.4.1.4 (e.g., amylosucrase), EC 2.4.1.5 (e.g., dextransucrase), EC 2.4.1.69 (e.g., galactoside 2-alpha-L-fucosyl transferase), EC 2.4.1.9 (e.g., inulosucrase), EC 2.7.1.17 (e.g., xylulokinase), EC 2.7.7.89 (formerly EC 3.1.4.15, e.g., [glutamine synthetase]-adenylyl-L-tyrosine phosphorylase), EC 2.7.9.4 (e.g., alpha glucan kinase) and EC 2.7.9.5 (e.g., phosphoglucan kinase).

In other embodiments a POI is a hydrolase enzyme, including, but not limited to, an EC 3 (hydrolase) enzyme selected from EC 3.1.X.X (e.g., an esterase), EC 3.1.1.1 (e.g., pectinase), EC 3.1.1.14 (e.g., chlorophyllase), EC 3.1.1.20 (e.g., tannase), EC 3.1.1.23 (e.g., glycerol-ester acylhydrolase), EC 3.1.1.26 (e.g., galactolipase), EC 3.1.1.32 (e.g., phospholipase A1), EC 3.1.1.4 (e.g., phospholipase A2), EC 3.1.1.6 (e.g., acetylesterase), EC 3.1.1.72 (e.g., acetylxylan esterase), EC 3.1.1.73 (e.g., feruloyl esterase), EC 3.1.1.74 (e.g., cutinase), EC 3.1.1.86 (e.g., rhamnogalacturonan acetylesterase), EC 3.1.1.87 (e.g., fumosin B1 esterase), EC 3.1.26.5 (e.g., ribonuclease P), EC 3.1.3.X (e.g., phosphoric monoester hydrolase), EC 3.1.30.1 (e.g., Aspergillus nuclease Si), EC 3.1.30.2 (e.g., Serratia marcescens nuclease), EC 3.1.3.1 (e.g., alkaline phosphatase), EC 3.1.3.2 (e.g., acid phosphatase), EC 3.1.3.8 (e.g., 3-phytase), EC 3.1.4.1 (e.g., phosphodiesterase I), EC 3.1.4.11 (e.g., phosphoinositide phospholipase C), EC 3.1.4.3 (e.g., phospholipase C), EC 3.1.4.4 (e.g., phospholipase D), EC 3.1.6.1 (e.g., arylsufatase), EC 3.1.8.2 (e.g., diisopropyl-fluorophosphatase), EC 3.2.1.10 (e.g., oligo-1,6-glucosidase), EC 3.2.1.101 (e.g., mannan endo-1,6-alpha-mannosidase), EC 3.2.1.11 (e.g., alpha-1,6-glucan-6-glucanohydrolase), EC 3.2.1.131 (e.g., xylan alpha-1,2-glucuronosidase), EC 3.2.1.132 (e.g., chitosan N-acetylglucosaminohydrolase), EC 3.2.1.139 (e.g., alpha-glucuronidase), EC 3.2.1.14 (e.g., chitinase), EC 3.2.1.151 (e.g., xyloglucan-specific endo-beta-1,4-glucanase), EC 3.2.1.155 (e.g., xyloglucan-specific exo-beta-1,4-glucanase), EC 3.2.1.164 (e.g., galactan endo-1,6-beta-galactosidase), EC 3.2.1.17 (e.g., lysozyme), EC 3.2.1.171 (e.g., rhamnogalacturonan hydrolase), EC 3.2.1.174 (e.g., rhamnogalacturonan rhamnohydrolase), EC 3.2.1.2 (e.g., beta-amylase), EC 3.2.1.20 (e.g., alpha-glucosidase), EC 3.2.1.22 (e.g., alpha-galactosidase), EC 3.2.1.25 (e.g., beta-mannosidase), EC 3.2.1.26 (e.g., beta-fructofuranosidase), EC 3.2.1.37 (e.g., xylan 1,4-beta-xylosidase), EC 3.2.1.39 (e.g., glucan endo-1,3-beta-D-glucosidase), EC 3.2.1.40 (e.g., alpha-L-rhamnosidase), EC 3.2.1.51 (e.g., alpha-L-fucosidase), EC 3.2.1.52 (e.g., beta-N-Acetylhexosaminidase), EC 3.2.1.55 (e.g., alpha-N-arabinofuranosidase), EC 3.2.1.58 (e.g., glucan 1,3-beta-glucosidase), EC 3.2.1.59 (e.g., glucan endo-1,3-alpha-glucosidase), EC 3.2.1.67 (e.g., galacturan 1,4-alpha-galacturonidase), EC 3.2.1.68 (e.g., isoamylase), EC 3.2.1.7 (e.g., 1-beta-D-fructan fructanohydrolase), EC 3.2.1.74 (e.g., glucan 1,4-O-glucosidase), EC 3.2.1.75 (e.g., glucan endo-1,6-beta-glucosidase), EC 3.2.1.77 (e.g., mannan 1,2-(1,3)-alpha-mannosidase), EC 3.2.1.80 (e.g., fructan beta-fructosidase), EC 3.2.1.82 (e.g., exo-poly-alpha-galacturonosidase), EC 3.2.1.83 (e.g., kappa-carrageenase), EC 3.2.1.89 (e.g., arabinogalactan endo-1,4-beta-galactosidase), EC 3.2.1.91 (e.g., cellulose 1,4-beta-cellobiosidase), EC 3.2.1.96 (e.g., mannosyl-glycoprotein endo-beta-N-acetylglucosaminidase), EC 3.2.1.99 (e.g., arabinan endo-1,5-alpha-L-arabinanase), EC 3.4.X.X (e.g., peptidase), EC 3.4.11.X (e.g., aminopeptidase), EC 3.4.11.1 (e.g., leucyl aminopeptidase), EC 3.4.11.18 (e.g., methionyl aminopeptidase), EC 3.4.13.9 (e.g., Xaa-Pro dipeptidase), EC 3.4.14.5 (e.g., dipeptidyl-peptidase IV), EC 3.4.16.X (e.g., serine-type carboxypeptidase), EC 3.4.16.5 (e.g., carboxypeptidase C), EC 3.4.19.3 (e.g., pyroglutamyl-peptidase I), EC 3.4.21.X (e.g., serine endopeptidase), EC 3.4.21.1 (e.g., chymotrypsin), EC 3.4.21.19 (e.g., glutamyl endopeptidase), EC 3.4.21.26 (e.g., prolyl oligopeptidase), EC 3.4.21.4 (e.g., trypsin), EC 3.4.21.5 (e.g., thrombin), EC 3.4.21.63 (e.g., oryzin), EC 3.4.21.65 (e.g., thermomycolin), EC 3.4.21.80 (e.g., streptogrisin A), EC 3.4.22.X (e.g., cysteine endopeptidase), EC 3.4.22.14 (e.g., actinidain), EC 3.4.22.2 (e.g., papain), EC 3.4.22.3 (e.g., ficain), EC 3.4.22.32 (e.g., stem bromelain), EC 3.4.22.33 (e.g., fruit bromelain), EC 3.4.22.6 (e.g., chymopapain), EC 3.4.23.1 (e.g., pepsin A), EC 3.4.23.2 (e.g., pepsin B), EC 3.4.23.22 (e.g., endothiapepsin), EC 3.4.23.23 (e.g., mucorpepsin), EC 3.4.23.3 (e.g., gastricsin), EC 3.4.24.X (e.g., metalloendopeptidase), EC 3.4.24.39 (e.g., deuterolysin), EC 3.4.24.40 (e.g., serralysin), EC 3.5.1.1 (e.g., asparaginase), EC 3.5.1.11 (e.g., penicillin amidase), EC 3.5.1.14 (e.g., N-acyl-aliphatic-L-amino acid amidohydrolase), EC 3.5.1.2 (e.g., L-glutamine amidohydrolase), EC 3.5.1.28 (e.g., N-acetylmuramoyl-L-alanine amidase), EC 3.5.1.4 (e.g., amidase), EC 3.5.1.44 (e.g., protein-L-glutamine amidohydrolase), EC 3.5.1.5 (e.g., urease), EC 3.5.1.52 (e.g., peptide-N(4)-(N-acetyl-beta-glucosaminyl)asparagine amidase), EC 3.5.1.81 (e.g., N-Acyl-D-amino-acid deacylase), EC 3.5.4.6 (e.g., AMP deaminase) and EC 3.5.5.1 (e.g., nitrilase).

In other embodiments a POI is a lyase enzyme, including, but not limited to, an EC 4 (lyase) enzyme selected from EC 4.1.2.10 (e.g., mandelonitrile lyase), EC 4.1.3.3 (e.g., N-acetylneuraminate lyase), EC 4.2.1.1 (e.g., carbonate dehydratase), EC 4.2.2.—(e.g., rhamnogalacturonan lyase), EC 4.2.2.10 (e.g., pectin lyase), EC 4.2.2.22 (e.g., pectate trisaccharide-lyase), EC 4.2.2.23 (e.g., rhamnogalacturonan endolyase) and EC 4.2.2.3 (e.g., mannuronate-specific alginate lyase).

In certain other embodiments a POI is an isomerase enzyme, including, but not limited to, an EC 5 (isomerase) enzyme selected from EC 5.1.3.3 (e.g., aldose 1-epimerase), EC 5.1.3.30 (e.g., D-psicose 3-epimerase), EC 5.4.99.11 (e.g., isomaltulose synthase) and EC 5.4.99.15 (e.g., (1→4)-α-D-glucan 1-α-D-glucosylmutase).

In yet other embodiments, a POI is a ligase enzyme, including, but not limited to, an EC 6 (ligase) enzyme selected from EC 6.2.1.12 (e.g., 4-coumarate:coenzyme A ligase) and EC 6.3.2.28 (e.g., L-amino-acid alpha-ligase).

These and other aspects and embodiments of the present strains and methods will be apparent to the skilled person in view of the present description and the following Examples.

### VII. Fermentation

In certain embodiments, the disclosure provides methods for producing a protein of interest comprising fermenting a filamentous fungal cell, wherein the fungal cell secrets the protein of interest. In other embodiments, the disclosure provides methods for producing increased amounts of ethanol in a yeast strain. In general, fermentation methods well known in the art are used to ferment the fungal cells. In some embodiments, the fungal cells are grown under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within batch cultures, cells progress through a static lag phase to a high growth log phase and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Certain embodiments of the instant disclosure are related to fermentation procedures for culturing fungi. Fermentation procedures for production of cellulase enzymes are known in the art. For example, cellulase enzymes can be produced either by solid or submerged culture, including batch, fed-batch and continuous-flow processes. Culturing is generally accomplished in a growth medium comprising an aqueous mineral salts medium, organic growth factors, a carbon and energy source material, molecular oxygen, and, of course, a starting inoculum of the filamentous fungal host to be employed.

In addition to the carbon and energy source, oxygen, assimilable nitrogen, and an inoculum of the microorganism, it is necessary to supply suitable amounts in proper proportions of mineral nutrients to assure proper microorganism growth, maximize the assimilation of the carbon and energy source by the cells in the microbial conversion process, and achieve maximum cellular yields with maximum cell density in the fermentation media.

The composition of the aqueous mineral medium can vary over a wide range, depending in part on the microorganism and substrate employed, as is known in the art. The mineral media should include, in addition to nitrogen, suitable amounts of phosphorus, magnesium, calcium, potassium, sulfur, and sodium, in suitable soluble assimilable ionic and combined forms, and also present preferably should be certain trace elements such as copper, manganese, molybdenum, zinc, iron, boron, and iodine, and others, again in suitable soluble assimilable form, all as known in the art.

The fermentation reaction is an aerobic process in which the molecular oxygen needed is supplied by a molecular oxygen-containing gas such as air, oxygen-enriched air, or even substantially pure molecular oxygen, provided to maintain the contents of the fermentation vessel with a suitable oxygen partial pressure effective in assisting the microorganism species to grow in a thriving fashion.

The microorganisms also require a source of assimilable nitrogen. The source of assimilable nitrogen can be any nitrogen-containing compound or compounds capable of releasing nitrogen in a form suitable for metabolic utilization by the microorganism. While a variety of organic nitrogen source compounds, such as protein hydrolysates, can be employed, usually cheap nitrogen-containing compounds such as ammonia, ammonium hydroxide, urea, and various ammonium salts such as ammonium phosphate, ammonium sulfate, ammonium pyrophosphate, ammonium chloride, or various other ammonium compounds can be utilized. Ammonia gas itself is convenient for large scale operations, and can be employed by bubbling through the aqueous ferment (fermentation medium) in suitable amounts. At the same time, such ammonia can also be employed to assist in pH control.

The pH range in the aqueous microbial ferment (fermentation admixture) should be in the exemplary range of about 2.0 to 8.0. With filamentous fungi, the pH normally is within the range of about 2.5 to 8.0; with Trichoderma reesei, the pH normally is within the range of about 3.0 to 7.0. Preferences for pH range of microorganisms are dependent on the media employed to some extent, as well as the particular microorganism, and thus change somewhat with change in media as can be readily determined by those skilled in the art.

Preferably, the fermentation is conducted in such a manner that the carbon-containing substrate can be controlled as a limiting factor, thereby providing good conversion of the carbon-containing substrate to cells and avoiding contamination of the cells with a substantial amount of unconverted substrate. The latter is not a problem with water-soluble substrates, since any remaining traces are readily washed off. It may be a problem, however, in the case of non-water-soluble substrates, and require added product-treatment steps such as suitable washing steps.

As described above, the time to reach this level is not critical and may vary with the particular microorganism and fermentation process being conducted. However, it is well known in the art how to determine the carbon source concentration in the fermentation medium and whether or not the desired level of carbon source has been achieved.

The fermentation can be conducted as a batch or continuous operation, fed batch operation is much to be preferred for ease of control, production of uniform quantities of products, and most economical uses of all equipment.

If desired, part or all of the carbon and energy source material and/or part of the assimilable nitrogen source such as ammonia can be added to the aqueous mineral medium prior to feeding the aqueous mineral medium to the fermenter.

Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermenter, cell density measurable by dry cell weights, light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible.

In either a batch, or the preferred fed batch operation, all equipment, reactor, or fermentation means, vessel or container, piping, attendant circulating or cooling devices, and the like, are initially sterilized, usually by employing steam such as at about 121° C. for at least about 15 minutes. The sterilized reactor then is inoculated with a culture of the selected microorganism in the presence of all the required nutrients, including oxygen, and the carbon-containing substrate. The type of fermenter employed is not critical.

The collection and purification of proteins from the fermentation broth can also be done by procedures known to one of skill in the art. The fermentation broth will generally contain cellular debris, including cells, various suspended solids and other biomass contaminants, as well as the desired cellulase enzyme product, which are preferably removed from the fermentation broth by means known in the art.

Suitable processes for such removal include conventional solid-liquid separation techniques such as, e.g., centrifugation, filtration, dialysis, microfiltration, rotary vacuum filtration, or other known processes, to produce a cell-free filtrate. It may be preferable to further concentrate the fermentation broth or the cell-free filtrate prior to crystallization using techniques such as ultrafiltration, evaporation or precipitation.

Precipitating the proteinaceous components of the supernatant or filtrate may be accomplished by means of a salt, e.g., ammonium sulfate, followed by purification by a variety of chromatographic procedures, e.g., ion exchange chromatography, affinity chromatography or similar art recognized procedures.

### VIII. Exemplary Embodiments

Non-limiting embodiments of the disclosure include, but are not limited to:

1. A variant (or modified) strain of filamentous fungus derived from a parental filamentous fungus strain comprising a gene encoding a native GEF1 protein, wherein the variant strain comprises a genetic modification which disrupts or deletes the gene encoding the native GEF1 protein, wherein the variant strain comprises an enhanced protein productivity phenotype relative to the parental strain when cultivated under the same conditions.

2. The variant strain of embodiment 1, wherein the enhanced protein productivity phenotype is selected from the group consisting of increased protein productivity, increased total protein productivity, increased volumetric productivity, increased carbon conversion efficiency and increased specific productivity.

3. The variant strain of embodiment 1, wherein the filamentous fungus is a Pezizomycotina.

4. The variant strain of embodiment 1, wherein the filamentous fungus is selected from the group consisting of a Trichoderma sp. strain, Aspergillus sp. strain, Fusarium sp. strain, Penicillium sp. strain, a Candida sp. strain, Chrysosporium sp. strain, Cephalosporium sp. strain, Talaromyces sp. strain, Neurospora sp. strain and Myceliophthora sp. strain.

5. The variant strain of embodiment 1, comprising an increased protein productivity phenotype relative to the parental strain when the variant and parental strains are fermented under the same conditions at 25° C.

6. The variant strain of embodiment 1, comprising an increased protein productivity phenotype relative to the parental strain when the variant and parental strains are fermented under the same conditions at 26° C.

7. The variant strain of embodiment 1, comprising an increased protein productivity phenotype relative to the parental strain when the variant and parental strains are fermented under the same conditions at 27° C.

8. The variant strain of embodiment 1, comprising an increased protein productivity phenotype relative to the parental strain when the variant and parental strains are fermented under the same conditions at 28° C.

9. The variant strain of embodiment 1, comprising a gene encoding an endogenous protein of interest (POI).

10. The variant strain of embodiment 9, wherein the endogenous POI is a lignocellulosic degrading enzyme.

11. The variant strain of embodiment 1, comprising an expression construct encoding a heterologous protein of interest (POI).

12. The variant strain of embodiment 11, wherein the heterologous POI is selected from an enzyme, an antibody or a fragment thereof, a receptor protein and a peptide.

13. The variant strain of embodiment 12, wherein the heterologous POI is an enzyme selected from the group consisting of oxidoreductases, transferases, hydrolases, lyases, isomerases and ligases.

14. The variant strain of embodiment 12, wherein the heterologous POI is an enzyme selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lyases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannanases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, and hexose oxidases.

15. A protein of interest produced by the variant strain of embodiment 1.

16. The variant strain of embodiment 1, wherein the encoded GEF1 protein comprises at least 70% sequence identity to SEQ ID NO: 2.

17. The variant strain of embodiment 1, wherein the encoded GEF1 protein comprises at least 90% sequence identity to the GEF1 domain of SEQ ID NO: 5.

18. The variant strain of embodiment 1, wherein the gene encoding the GEF1 protein comprises at least 70% sequence identity to the GEF1 polynucleotide sequence of SEQ ID NO: 1.

19. The variant strain of embodiment 1, wherein the gene encoding the GEF1 protein comprises at least 70% sequence identity to the open reading frame (ORF) sequence of SEQ ID NO: 3.

20. The variant strain of embodiment 1, wherein the gene encoding the GEF1 protein hybridizes with a GEF1 gene of SEQ ID NO: 1, or the GEF1 ORF of SEQ ID NO: 3, under medium to stringent hybridization conditions.

21. The variant strain of embodiment 1, wherein the genetic modification truncates at least the first 20 amino acid residues of the GEF1 N-terminus, or truncates at least the last 20 amino acid residues of the GEF1 C-terminus.

22. The variant strain of embodiment 1, wherein the genetic modification truncates at least the first 200 amino acid residues of the GEF1 N-terminus, or truncates at least the last 200 amino acid residues of the GEF1 C-terminus.

23. The variant strain of embodiment 1, wherein the genetic modification truncates at least the first 500 amino acid residues of the GEF1 N-terminus, or truncates at least the last 500 amino acid residues of the GEF1 C-terminus.

24. The variant strain of embodiment 1, wherein the genetic modification truncates at least 700 to 750 amino acid residues of the GEF1 C-terminus.

25. The variant strain of embodiment 1, wherein the gene encoding the GEF1 protein is deleted.

26. A method for producing an increased amount of an endogenous protein of interest (POI) in a modified filamentous fungal strain comprising (a) genetically modifying a filamentous fungal strain by disrupting or deleting the native GEF1 gene, and (b) fermenting the modified strain under suitable conditions for the production of the POI, wherein the modified strain produces an increased amount of the POI relative to the parental strain when cultivated under the same conditions at 25° C..

27. The method of embodiment 26, wherein the filamentous fungus is a Pezizomycotina.

28. The method of embodiment 26, wherein the filamentous fungus is selected from the group consisting of a Trichoderma sp. strain, Aspergillus sp. strain, Fusarium sp. strain, Penicillium sp. strain, a Candida sp. strain, Chrysosporium sp. strain, Cephalosporium sp. strain, Talaromyces sp. strain, Neurospora sp. strain and Myceliophthora sp. strain.

29. The method of embodiment 26, wherein the modified strain produces an increased amount of the POI relative to the parental strain when cultivated under the same conditions at 26° C..

30. The method of embodiment 26, wherein the modified strain produces an increased amount of the POI relative to the parental strain when cultivated under the same conditions at 27° C.

31. The method of embodiment 26, wherein the modified strain produces an increased amount of the POI relative to the parental strain when cultivated under the same conditions at 28° C.

32. The method of embodiment 26, wherein the endogenous POI is a lignocellulosic degrading enzyme.

33. The method of embodiment 26, wherein the modified strain further comprises an expression construct encoding a heterologous POI.

34. The method of embodiment 26, wherein the encoded GEF1 protein comprises at least 70% sequence identity to SEQ ID NO: 2.

35. The method of embodiment 26, wherein the encoded GEF1 protein comprises at least 90% sequence identity to the GEF1 domain of SEQ ID NO: 5.

36. The method of embodiment 26, wherein the gene encoding the GEF1 protein comprises at least 70% sequence identity to the GEF1 polynucleotide sequence of SEQ ID NO: 1.

37. The method of embodiment 26, wherein the gene encoding the GEF1 protein comprises at least 70% sequence identity to the open reading frame (ORF) sequence of SEQ ID NO: 3.

38. The method of embodiment 26, wherein the gene encoding the GEF1 protein hybridizes with a GEF1 gene of SEQ ID NO: 1, or a GEF1 ORF of SEQ ID NO: 3, under medium to stringent hybridization conditions.

39. The method of embodiment 26, wherein the genetic modification truncates at least the first 20 amino acid residues of the GEF1 N-terminus, or truncates at least the last 20 amino acid residues of the GEF1 C-terminus.

40. The method of embodiment 26, wherein the genetic modification truncates at least the first 200 amino acid residues of the GEF1 N-terminus, or truncates at least the last 200 amino acid residues of the GEF1 C-terminus.

41. The method of embodiment 26, wherein the genetic modification truncates at least the first 500 amino acid residues of the GEF1 N-terminus, or truncates at least the last 500 amino acid residues of the GEF1 C-terminus.

42. The method of embodiment 26, wherein the genetic modification truncates at least 700 to 750 amino acid residues of the GEF1 C-terminus.

43. The method of embodiment 26, wherein the gene encoding the GEF1 protein is deleted.

44. A method for producing increased amounts of a heterologous protein of interest (POI) in a modified filamentous fungal strain comprising (a) genetically modifying a filamentous fungal strain by disrupting or deleting the native GEF1 gene and by introducing an expression cassette encoding a heterologous POI, and (b) fermenting the modified strain under suitable conditions for the production of the heterologous POI, wherein the modified strain produces an increased amount of the POI relative to the parental strain when cultivated under the same conditions at 25° C..

45. The method of embodiment 44, wherein the filamentous fungus is a Pezizomycotina.

46. The method of embodiment 44, wherein the filamentous fungus is selected from the group consisting of a Trichoderma sp. strain, Aspergillus sp. strain, Fusarium sp. strain, Penicillium sp. strain, a Candida sp. strain, Chrysosporium sp. strain, Cephalosporium sp. strain, Talaromyces sp. strain, Neurospora sp. strain and Myceliophthora sp. strain.

47. The method of embodiment 44, wherein the modified strain produces an increased amount of the POI relative to the parental strain when cultivated under the same conditions at 26° C..

48. The method of embodiment 44, wherein the modified strain produces an increased amount of the POI relative to the parental strain when cultivated under the same conditions at 27° C.

49. The method of embodiment 44, wherein the modified strain produces an increased amount of the POI relative to the parental strain when cultivated under the same conditions at 28° C.

50. The method of embodiment 44, wherein the heterologous POI is selected from an enzyme, an antibody or a fragment thereof, a receptor protein and a peptide.

51. The method of embodiment 44, wherein the heterologous POI is an enzyme selected from the group consisting of oxidoreductases, transferases, hydrolases, lyases, isomerases and ligases.

52. The method of embodiment 44, wherein the heterologous POI is an enzyme selected from the group consisting of acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lyases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannanases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, and hexose oxidases.

53. A protein of interest produced by the method of embodiment 26 or embodiment 44.

54. A method for producing an increased amount of ethanol in a yeast fermentation process comprising (a) genetically modifying a yeast strain by deleting or disrupting the native GEF1 gene, and (b) fermenting the modified strain under suitable conditions for the production of ethanol, wherein the modified strain produces an increased amount of ethanol at the end of fermentation (EOF) relative to the amount of ethanol produced by the parental strain at the EOF when cultivated under the same conditions.

55. A variant yeast strain derived from a parental strain comprising a gene encoding a GEF1 protein homologue, wherein the variant strain comprises a genetic modification which deletes or disrupts the gene encoding the GEF1 protein homologue, wherein the variant strain comprises an increased ethanol productivity phenotype relative to the parental strain when cultivated under the same conditions

### EXAMPLES

Certain aspects of the present disclosure may be further understood in light of the following examples, which should not be construed as limiting. Modifications to materials and methods will be apparent to those skilled in the art.

### Example 1

### Identifying Mutant Trichoderma Strains Comprising Enhanced Protein Productivity Phenotypes at Elevated Cultivation Temperatures

A Trichoderma reesei whole cellulase (T4) strain was serially propagated under selective conditions to isolate mutants capable of high temperature protein production, without adversely affecting specific productivity. A mutant T. reesei strain named “T4-E1” was identified and isolated with a similar specific productivity (Qp) at 28° C., relative to the parental T. reesei T4 strain at 25° C. For example, the T4 parental strain was sporulated on BIRD agar and 1×10⁷ spores/mL were collected from the agar plate, suspended in water and treated with 0.15 mg/mL of 1-methyl-3-nitro-1-nitrosoguanidine (Sigma 112, 994-1) for two (2) hours at room temperature until only 1% of the spores remained viable. The spores were inoculated into an evolution media containing 0.5% of either microcrystalline cellulose (EMCOCEL, JRS Pharma, Rosenburg, Germany), carboxymethylcellulose (CMC; Sigma-Aldrich C5678, St. Louis, MO), or acid swollen cellulose (e.g., for preparation see, Wood, 1988) as the sole carbon source. Also, per liter ammonium sulfate (4 g), sodium phosphate monobasic (4.5 g), magnesium sulfate heptahydrate (1 g), calcium chloride dihydrate (1 g) and 2.5 ml of a 400× trace element solution.

More specifically, in a first method, about one (1) million chemically mutated spores were inoculated into a two-hundred fifty (250) ml dented bottom flask containing the evolution medium described above with Avicel® as the sole carbon source. The flask was incubated at 180 rpm, 31° C. for five (5) days. At this time, a 10% volume/volume transfer was made to a second identical flask which was incubated in the same manner. The serial transfer continued for eleven (11) passages (P) as follows: P1=five (5) days, P2=five (5) days, P3=four (4) days, P4=four (4) days, P5=four (4) days, P6=four (4) days, P7=four (4) days, P8=four (4) days, P9=three (3) days, P10=three (3) days and P11=two (2) days. The P11 broth from the P11 shake flasks was centrifuged at 4,000 rpm for ten (10) minutes. The supernatant was discarded and the cells were suspended in water and plated onto BIRD medium.

Individual colony forming units were evaluated for total protein BCA (Product #23228, Thermo Scientific, Rockford, IL) after incubation in a slow-release lactose micro titer plate (srMTP; e.g., see PCT Publication No. WO2014/047520) for four (4) days, 31° C., 200 rpm, and 80% humidity. Thus, mutant strains producing an equivalent amount of total protein at 31° C. relative to the amount of total protein produced by the parental strain at 25° C., were further evaluated for high temperature protein production in fermentors.

In a second method, mutated spores of the T. reesei T4 parental strain were encapsulated in water and oil emulsion droplets using methods described in Bachmann et al. (2013). The droplets contained evolution medium with either 0.5% microcrystalline cellulose (EMCOCEL, JRS Pharma, Rosenburg, Germany), carboxymethylcellulose (Sigma-Aldrich C5678, St. Louis, MO), or acid swollen cellulose (Wood, 1988) as the sole carbon source. Also, per liter ammonium sulfate (4 g), sodium phosphate monobasic (4.5 g), magnesium sulfate heptahydrate (1 g), calcium chloride dihydrate (1 g), and 2.5 ml of a 400× trace element solution. The droplets were incubated at 31° C. in a tube for three (3) days at which time the emulsion was disrupted using methods described in Bachmann et al. (2013). The cells were recovered, sporulated on agar plates, re-encapsulated and incubated at 31° C. for three (3) days. This process was repeated 10 times. After the final transfer, the cells were suspended in water and plated onto BIRD medium. Individual colony forming units were evaluated for total BCA protein (Product #23228, Thermo Scientific, Rockford, IL) after incubation in a srMTP lactose plate (PCT Publication No. WO2014/047520) for four (4) days, 31° C., 200 rpm, and 80% humidity. Mutant strains producing an equivalent amount of total protein at 31° C. relative to the amount of total protein produced by the parental strain at 25° C., were further evaluated for high temperature protein production in fermentors. Thus, as generally set forth above and presented below, a mutant T. reesei (T4) strain named “T4-E1” was identified as a high temperature mutant capable of optimal protein production at 28° C. compared to (vis-à-vis) the optimal protein production of the parental T4 strain at 25° C.

### Example 2

### Characteristics of the Mutant Trichoderma Strain Comprising A Mutated GEF1 Gene

The gene that was mutated in the Trichoderma variant (mutant) strain described above in Example 1 resides at scaffold position 3:1532639-1538781 in the wild-type T. reesei QM6a v2.0 genome sequence assembly, available at the Joint Genomes Institute (JGI) website (genome.jgi.doe.gov). For example, the deduced amino acid sequence (PID:120482; SEQ ID NO: 2) includes a region of sequence homology (e.g., SEQ ID NO: 4) with the Rho/Rac/Cdc42-like guanine nucleotide exchange factor domain (GEF1 domain; Pfam PF00621, also called Dbl-homologous or DH domain). This homologous region is at amino acid residue positions 1,242-1,454 in the deduced amino acid sequence (i.e., PID:120482; SEQ ID NO: 2). The Rho family GTPases regulate many different cellular processes, wherein such GTPases are activated through release of bound GDP and subsequent binding of GTP, which is catalysed by guanine exchange factors in the GEF1 family. The mutation in the variant T4-E 1 strain replaces a glutamate codon (Glu (E); amino acid position 1,245) with a pre-mature stop codon, thereby truncating the protein near the beginning of the GEF1 domain (e.g., see FIG. 3A-3C).

Likewise, the deduced amino acid sequence (PID:120482; SEQ ID NO: 2) also comprises homology to the BAR or AH/BAR domain (e.g., SEQ ID NO: 5; Pfam PF03114) at residue positions 1,576-1,677 of SEQ ID NO: 2. The AH/BAR domain is found in a range of proteins. In some cases, it has been shown to interact with membranes, dimerize or to bind Arf and Rho family GTPases, including ARF1, a small GTPase involved in vesicle budding at the Golgi complex. The AH/BAR domain (SEQ ID NO: 5) is not observed in the other four (4) T. reesei GEF1 domain proteins.

Unlike some other GEF1 domain proteins, no Plekstrin homology domain (PH domain, Pfam PF00169) is found in SEQ ID NO: 2 (PID:120482). The PH domain can bind phosphatidylinositol in membranes and the beta/gamma subunits of heterotrimeric G proteins or protein kinase C, and thereby is associated with signal transduction pathways. There are four (4) other deduced protein sequences found in the T. reesei QM6a genome that contain GEF1 domains, of which two (2) also contain PH domains.

In addition, the deduced amino acid sequence (PID:120482; SEQ ID NO: 2) includes a C-terminal sequence (e.g., SEQ ID NO: 6) at residue positions 1,918-1,990 of SEQ ID NO: 2 that is highly conserved with some other filamentous fungal GEF1 domain proteins, including Fusarium fujikuroi, Fusarium graminearum, Thielavia terrestris, Podospora anserina, Penicillium rubens and Aspergillus niger. However, this C-terminal sequence domain (SEQ ID NO: 6) is not in the other four (4) GEF1 domain proteins identified in the T. reesei genome.

### Example 3

### Inactivation of the GEF1 Gene in a Trichoderma Strain by Insertion of Pyr2 Gene

Inactivation of the GEF1 gene (JGI T. reesei v2.0 scaffold 3:1532639-1538781) encoding a GEF1 protein of SEQ ID NO: 2 (PID:120482) was performed in a T. reesei strain using a Cas9-based method.

More particularly, purified Cas9 protein and Modified EZ tracrRNA were purchased from Synthego Corporation (Redwood City, CA) and the modified crRNA was synthesized by Synthego with the following sequence at the 5′ end (TS3; SEQ ID NO: 7) that is specific to a target site (TS3) within the T. reesei GEF1 gene.

(SEQ ID NO: 7)
TS3: CAUUCAUCCAAGAAUCCGAG

The Cas9 target site (TS) in the GEF1 gene, determined by the above RNA sequence (SEQ ID NO: 7) is at nucleotide positions 3,659-3,678 in the coding sequence, which is close to the mutation at nucleotide position 3,733 observed in the T4-E1 mutant strain (Example 2). The tracrRNA and crRNA were annealed to form guide RNA (gRNA) and then combined with Cas9-2NLS to form a ribonucleoprotein complex (Cas9:RNP) according to the manufacturer's directions. Before use, the Cas9:RNP's were mixed with Lipofectamine CRISPR-MAX, purchased from ThermoFisher Scientific, Inc. (Waltham, MA).

A linear DNA fragment containing the T. reesei pyr2 gene with native promoter and terminator sequences and flanked by 492 bp of T. reesei repeat (SEQ ID NO: 18) was amplified by PCR using primers AL950 (SEQ ID NO: 8) and AL952 (SEQ ID NO: 9) primers. The resulting PCR product was purified using a Qiagen QIAquick PCR purification kit.

(SEQ ID NO: 8)
AL950: CCTAACTAACGTCTGACATCG
(SEQ ID NO: 9)
AL952: CGTACCATTTGACTGATACGATG

Protoplasts of the T. reesei parental strain were transformed with the pyr2 PCR product plus the Cas9:RNP. Transformants were selected for uridine auxotrophy. Screening transformants for the desired insertion of pyr2 into the GEF1 gene was conducted by PCR using forward and reverse primer pairs RhoF1 (SEQ ID NO: 10) and RhoR1 (SEQ ID NO: 11), which primers amplify across the GEF1 Cas9

(SEQ ID NO: 10)
RhoF1: GAGCTAGACACGGGCGAAG
(SEQ ID NO: 11)
RhoR1: AGGAACCCTTCGTGAGCAAG

Insertion of pyr2 increased the size of the PCR product from 755 bp to approximately 2.8 kb. The DNA sequence of the 2.8 kb PCR product from transformant named “ΔRho #22D” was determined by Sanger sequencing using the RhoF1 and RhoR1 primers, verifying insertion of pyr2 at the Cas9 cleavage site and disruption of the GEF1 coding sequence.

As presented in TABLE 1, the fermenter performance of the ΔRho #22D transformant was compared to the parental T4 strain and the mutant named T4-E 1 strain described in Example 1. For example, the final specific productivity (Qp) rates of the T4, T4-E1 and T4-ΔRho #22D strains at 28° C. are shown in TABLE 1 as percentages (%) relative to the final Qp rate of the control strain T4 at 25° C.

TABLE 1
FINAL Qp RATE (g/gDCW/hr) AS
PERCENT OF CONTROL (T4 25° C.)

| | | Final Qp Rate |
|---|---|---|
| | Temperature | % of control |
| Strain name | (° C.) | (T4 25° C.) |
| T4 | 25 | 100 |
| T4 | 28 | 68 |
| T4-E1 | 28 | 98 |
| T4-ΔRho#22D | 28 | 107 |

### Example 4

### Inactivation of the GEF1 Gene by Partial Deletion in a Trichoderma Strain Comprising a Heterologous Cellulase Expression Construct

As described in the instant example, a Trichoderma strain named “T4-DXST” was derived/constructed from a parental T. reesei T4 strain, wherein the native cellulase genes encoding the CBHI, CBHII, EGI and EGII proteins were inactivated by deletion or disruption (i.e., using routine molecular biology techniques known by one of skill in the art), and transformed with copies of the gene encoding Staphylotrichum coccosporum endoglucanase 1 (STCE1) placed under the control of the high efficiency cbhI promoter.

Methods for transformation of T. reesei with exogenously added plasmid DNA have been published (Penttila et al., 1986; Gruber et al., 1990; Smith et al., 1991). Stable transformants arise by integration of plasmid DNA into the chromosomes of the host. Integration can be at sites in the genome that have homology with a region of the plasmid DNA or can be at non-homologous sites.

Inactivation of the gene encoding GEF1 in a parental T. reesei strain comprising copies of the introduced STCE1 cellulase construct (e.g., see U.S. Pat. No. 7,595,182) was performed using a Cas9-based method. More particularly, purified Cas9-2NLS protein and Modified EZ tracrRNA were purchased from Synthego Corporation (Redwood City, CA). Modified crRNAs were synthesized by Synthego with the following RNA sequences at the 5′ end that are specific to target sites 3 and 4 (TS3/TS4) within the T. reesei GEF1 gene.

(SEQ ID NO: 7)
TS3: CAUUCAUCCAAGAAUCCGAG
(SEQ ID NO: 12)
TS4: AUUGUGGAGGAAAUCUACAA

The Cas9 target sites (TS3/TS4) in the GEF1 gene determined by the above RNA sequences are at nucleotide positions 3,659-3,678 and at nucleotide positions 3,775-3794 in the coding sequence, which is close to the mutation at nucleotide position 3,733 observed in the T4-E1 mutant strain. The tracrRNA and crRNA were annealed to form guide RNA (gRNA) and then combined with Cas9-2NLS to form a ribonucleoprotein complex (Cas9:RNP), according to the manufacturer's directions. Before use, the Cas9:RNP's were mixed with Lipofectamine CRISPR-MAX purchased from ThermoFisher Scientific, Inc. (Waltham, MA).

A linear DNA fragment containing a bacterial hygromycin phosphotransferase gene (SEQ ID NO: 13) operably linked to the Neurospora crassa cpcl promoter (SEQ ID NO: 14) and Aspergillus nidulans trpC terminator (SEQ ID NO: 15) was amplified by PCR using primers HygF1 (SEQ ID NO: 16) and HygR1 (SEQ ID NO: 17).

(SEQ ID NO: 16)
HygF1: TGGCTTTCCGTCTCCATTG
(SEQ ID NO: 17)
HygR1: AGTGTACCTGTGCATTCTGGG

Protoplasts of the T. reesei parental strain were transformed with the linear DNA fragment and a mixture of both Cas9:RNP's (with both crRNAs). Transformants were selected for hygromycin resistance on appropriate agar medium. However, only smaller and slower growing transformed colonies were picked from the selective agar plates and were transferred to agar plates with non-selective medium. After growth on the non-selective medium, transformants were picked and spotted onto medium with hygromycin. Transformants that did not grow in the presence of hygromycin were chosen for further study. It was presumed that these transformants had exhibited hygromycin resistance transiently, and had subsequently lost the hygromycin-resistance DNA marker fragment without integration into the T. reesei genome. PCR with the RhoF1 (SEQ ID NO: 10) and RhoR1 (SEQ ID NO: 11) primers was used to screen the hygromycin-sensitive transformants.

Transformants were identified that gave a PCR product of approximately 630 bp, as expected if a deletion between Cas9 target sites TS3 and TS4 had occurred. The size of this PCR product in wild-type cells is 756 bp. The PCR products were sequenced from each end using primers RhoF1 (SEQ ID NO: 10) and RhoR1 (SEQ ID NO: 11). Transformant #89 (t-AWC88) had a 125 bp deletion (i.e., nucleotide positions 3,668-3,791 in the coding sequence) between TS3 and TS4 with no insertion. The Trichoderma transformant #89 (t-AWC88) was subsequently fermented and compared (vis-à-vis) to the parental strain (t-AVT84) for STCE/DXST protein production at 25° C. and 28° C. as presented in TABLE 2.

TABLE 2
FINAL PROTEIN YIELD DURING PRODUCTION PHASE AS
PERCENT OF CONTROL STRAIN t-AVT84 AT 25° C.

| | | Final Yield (g/g) |
|---|---|---|
| | Temperature | % of Control |
| Strain Name | (° C.) | (t-AVT84 25° C.) |
| t-AVT84 | 25 | 100 |
| t-AVT84 | 28 | 58 |
| t-AWC88 | 25 | 111 |
| t-AWC88 | 26 | 106 |
| t-AWC88 | 27 | 106 |
| t-AWC88 | 28 | 78 |
| t-AWC88 | 30 | 55 |

More particularly, as shown above in TABLE 2, the T4-DXST daughter strain (t-AWC88) has at least a 2-3° C. higher protein production temperature optimum relative to the parental strain (t-AVT84).

### REFERENCES

PCT Application Serial No. PCT/US2019/27590
PCT Publication No. WO2011/153449
PCT Publication No. WO2011/153449
PCT Publication No. WO2012/145584
PCT Publication No. WO2012/145595
PCT Publication No. WO2016/100272
PCT Publication No. WO2016/100562
PCT Publication No. WO2016/100568
U.S. Pat. No. 6,022,725
U.S. Pat. No. 6,255,115.
U.S. Pat. No. 6,268,328
U.S. Pat. No. 7,595,182
U.S. Provisional Application Ser. No. 62/711,846, filed Jul. 30, 2018.
Altschul et al., J. Mol. Biol. 215:403-10, 1990.
Altschul et al., Meth. Enzymol. 266:460-80, 1996.
Ausubel et al., “Current Protocols in Molecular Biology”, Green Publishing Associates/Wiley Interscience, New York, 1994.
Bachmann et al., “Availability of public goods shapes the evolution of competing metabolic strategies”, PNAS, 110 (35): 14302-14307, 2013.
Boel et al., EMBO J 3:1581-1585, 1984.
Botstein and Shortle, Science 229: 4719, 1985.
Campbell et al., Curr. Genet., 16: 53-56, 1989.
Cao et al., Science, 9: 991-1001, 2000.
Devereux et al., Nucleic Acids Res. 12:387-395, 1984.
Gruber et al., 1990
Hakkinen et al., “Screening of candidate regulators for cellulase and hemicellulase production in Trichoderma reesei and identification of a factor essential for cellulase production”, Biotechnol Biofuels 7, 14, 2014.
Harkki et al., BioTechnol., 7: 596-603, 1989.
Harkki et al., Enzyme Microb. Technol., 13: 227-233, 1991.
Henikoff and Henikoff, Proc. Natl. Acad. Sci. USA 89:10915, 1989.
Higgins and Sharp, CABIOS 5:151-53, 1989.
Higuchi et al., Nucleic Acids Research 16: 7351, 1988.
Ho et al., Gene 77: 61, 1989.
Horton et al., Gene 77: 61, 1989.
Iglesias and Trautner, Molecular General Genetics 189: 73-76, 1983.
Joint Genomes Institute (JGI) website (www.genome.jgi.doe.gov).
Karlin et al., Proc. Natl. Acad. Sci. USA 90:5873-87, 1993.
Lo et al., Proceedings of the National Academy of Sciences USA 81: 2285, 1985.
Mullaney et al., MGG 199:37-45, 1985.
Needleman and Wunsch, J. Mol. Biol., 48:443, 1970.
Nunberg et al., Mol. Cell Biol. 4:2306, 1984.
Parish and Stoker, FEMS Microbiology Letters 154: 151-157, 1997.Pearson and Lipman, 1988
Penttila et al., “A versatile transformation system for the cellulolytic filamentous fungus Trichoderma reesei”, Gene 61, 155-164, 1987.
Perego, 1993, In A. L. Sonneshein, J. A. Hoch, and R. Losick, editors, Bacillus subtilis and Other Gram-Positive Bacteria, Chapter 42, American Society of Microbiology, Washington, D.C.
Sambrook et al., “Molecular Cloning, A Laboratory Manual”, 4th Edition, Cold Spring Harbor Laboratory Press, Cold Spring, New York, 2012.
Sarkar and Sommer, BioTechniques 8: 404, 1990.
Shimada, Meth. Mol. Biol. 57: 157; 1996.
Smith and Waterman, Adv. Appl. Math. 2:482, 1981.
Wood, “Preparation of crystalline, amorphous, and dyed cellulase substrates”, Methods in Enzymology, Vol. 160, pages 19-25, 1988.
Yelton et al., PNAS USA 81:1470-1474, 1984.
Youngman et al., 1983

1. A variant filamentous fungal cell derived from a parental cell comprising a gene encoding a GEF1 protein, wherein the variant cell comprises a genetic modification which disrupts or deletes the gene encoding the GEF1 protein, wherein the variant cell comprises an enhanced protein productivity phenotype relative to the parental cell when fermented under the same conditions.

2. The variant cell of claim 1, wherein the enhanced protein productivity phenotype is selected from the group consisting of increased protein productivity, increased total protein productivity, increased volumetric productivity, increased carbon conversion efficiency and increased specific productivity.

3. The variant cell of claim 1, comprising an increased protein productivity phenotype relative to the parental cell when the variant and parental cells are fermented under the same conditions at 26° C.

4. The variant cell of claim 1, comprising a gene encoding an endogenous protein of interest (POI) and/or comprising an expression cassette encoding a heterologous POI.

5. The variant cell of claim 4, wherein the endogenous POI is a lignocellulosic degrading enzyme.

6. The variant cell of claim 5, wherein the lignocellulosic degrading enzyme is selected from the group consisting of a cellobiohydrolase, a xylanase, an endoglucanase and a β-glucosidase.

7. The variant cell of claim 4, wherein the expression cassette encodes a heterologous POI selected from an enzyme, an antibody, a receptor and a peptide.

8. The variant cell of claim 7, wherein the enzyme is selected from the group consisting of oxidoreductases, transferases, hydrolases, lyases, isomerases and ligases.

9. A protein of interest produced by the variant cell of claim 1.

10. A method for producing an increased amount of an endogenous protein of interest (POI) in a modified filamentous fungal cell comprising:
(a) genetically modifying a parental filamentous fungal cell by disrupting or deleting the native GEF1 gene, and
(b) fermenting the modified cell under suitable conditions for the production of the POI,
wherein the modified cell produces an increased amount of the POI relative to the parental strain when fermented under the same conditions at 25° C..

11. The method of claim 10, wherein the endogenous POI is a lignocellulosic degrading enzyme.

12. The method of claim 11, wherein the lignocellulosic degrading enzyme is selected from the group consisting of a cellobiohydrolase, a xylanase, an endoglucanase and a β-glucosidase.

13. The method of claim 10, wherein the modified strain further comprises an expression construct encoding a heterologous POI.

14. A method for producing an increased amount of a heterologous protein of interest (POI) in a modified filamentous fungal cell comprising:
(a) genetically modifying a filamentous fungal cell by disrupting or deleting the native GEF1 gene and by introducing an expression cassette encoding a heterologous POI, and
(b) fermenting the modified cell under suitable conditions for the production of the heterologous POI, wherein the modified cell produces an increased amount of the POI relative to the parental strain when fermented under the same conditions at 25° C..

15. The method of claim 14, wherein the heterologous POI is selected from an enzyme, an antibody or a fragment thereof, a receptor protein and a peptide.

16. The method of claim 14, wherein the heterologous POI is an enzyme selected from the group consisting of oxidoreductases, transferases, hydrolases, lyases, isomerases and ligases.

17. A protein of interest produced by the method of claim 10 or claim 14.

18. A method for producing an increased amount of ethanol in a yeast fermentation process comprising:
(a) genetically modifying a yeast strain by deleting or disrupting the native GEF1 gene, and
(b) fermenting the modified strain under suitable conditions for the production of ethanol,
wherein the modified strain produces an increased amount of ethanol at the end of fermentation (EOF) relative to the amount of ethanol produced by the parental strain at the EOF when fermented under the same conditions.

19. A variant yeast strain derived from a parental strain comprising a gene encoding a GEF1 protein homologue, wherein the variant strain comprises a genetic modification which deletes or disrupts the gene encoding the GEF1 protein homologue, wherein the variant strain comprises an increased ethanol productivity phenotype relative to the parental strain when fermented under the same conditions.
